(19) United States
(12) Patent Application Publication
(10) Pub. No.: US 2009/0028886 A1
(43) Pub. Date: Jan. 29, 2009

(54) Carrier conjugates of gnrh-peptides

(75) Inventors: Martin F Bachmann, Seuzach (CH); Alma Fulurija, Schlieren (CH); Gary Jennings, Zurich (CH); Edwin Meijerink, Dunedin (NZ)

Correspondence Address: STERNE, KESSLER, GOLDSTEIN & FOX P.L.L.C., 1100 NEW YORK AVENUE, N.W., WASHINGTON, DC, 20005, US

(73) Assignee: CYTOS BIOTECHNOLOGY AG, Zurich-Schlieren (CH)

(21) Appl. No.: 11/659,221

(22) PCT Filed: Aug. 4, 2005
(86) PCT No.: PCT/EP05/53858

§ 371 Date: Feb. 1, 2008

Related U.S. Application Data
(60) Provisional application No. 60/598,581, filed on Aug. 4, 2004.
(60) Provisional application No. 60/673,396, filed on Apr. 21, 2005.

(51) Int. Cl. A61K 39/385; A61P 35/00

(52) U.S. Cl. 424/185.1

(57) ABSTRACT

The present invention is related to the fields of molecular biology, virology, immunology and medicine. The invention provides a composition comprising a virus like particle (VLP) and at least one GnRH peptide or fragment or variant thereof linked thereto.

The invention also provides a process for producing the composition. The compositions of the invention are useful in the production of vaccines for the treatment of GnRH-related diseases and conditions and to efficiently induce immune responses, in particular antibody responses. Furthermore, the compositions of the invention are particularly useful to efficiently induce self-specific immune responses within the indicated context.

### BACKGROUND OF THE INVENTION

1. Field of the Invention

The present invention is related to the fields of molecular biology, virology, immunology and medicine. The invention provides a composition comprising: a virus like particle (VLP) and at least one GnRH peptide, wherein the VLP and the at least one GnRH peptide are linked with one another.

The invention also provides a process for producing the composition of the invention. The compositions of the invention are useful in the production of vaccines for the treatment of GnRH associated diseases and conditions and to efficiently induce immune responses, in particular antibody responses. Furthermore, the compositions of the invention are particularly useful to efficiently induce self-specific immune responses within the indicated context.

2. Related Art

Gonadotropin Releasing Hormone (GnRH) is of central importance to the regulation of fertility. A number of important diseases are affected by gonadotropins and gonadal steroid hormones, particularly the gonadal steroids estrogen and testosterone. Such diseases include breast cancer, uterine and other gynecological cancers, endometriosis, uterine fibroids, prostate cancer and benign prostatic hypertrophy, among others. In addition, androstenone, a precursor molecule of testosterone, is the main responsible steroid for the formation of disagreeable odour of meat of male, sexually mature pigs (boars), male cattle (bulls) and male sheep (rams), mainly pigs. Immunizations against antigens derived from GnRH have been reported.

It is usually difficult to induce antibody responses against self-antigens. One way to improve the efficiency of vaccination is to increase the degree of repetitiveness of the antigen applied. Unlike isolated proteins, viruses induce prompt and efficient immune responses in the absence of any adjuvant both with and without T-cell help (Bachmann and Zinkernagel, Ann. Rev. Immunol: 15:235-270 (1991)). Although viruses often consist of few proteins, they are able to trigger much stronger immune responses than their isolated components. For B-cell responses, it is known that one crucial factor for the immunogenicity of viruses is the repetitiveness and order of surface epitopes. Many viruses exhibit a quasi-crystalline surface that displays a regular array of epitopes which efficiently crosslinks epitope-specific immunoglobulins on B-cells (Bachmann and Zinkernagel, Immunol. Today 17:553-558 (1996)). This crosslinking of surface immunoglobulins on B cells is a strong activation signal that directly induces cell-cycle progression and the production of IgM antibodies. Further, such triggered B-cells are able to activate T helper cells, which in turn induce a switch from IgM to IgG antibody production in B cells and the generation of long-lived B cell memory—the goal of any vaccination (Bachmann and Zinkernagel, Ann. Rev. Immunol. 15:235-270 (1997)). Viral structure is even linked to the generation of anti-antibodies in autoimmune disease and as a part of the natural response to pathogens (see Fehr, T., et al., J. Exp. Med. 185:1785-1792 (1997)). Thus, antigens presented by a highly organized viral surface are able to induce strong antibody responses against the antigens.

As indicated, however, the immune system usually fails to produce antibodies against self-derived structures. For soluble antigens present at low concentrations, this is due to tolerance at the Th-cell level. Under these conditions, coupling the self-antigen to a carrier that can deliver T help may break tolerance. For soluble proteins present at high concentrations or membrane proteins at low concentration, B- and Th-cells may be tolerant. However, B-cell tolerance may be reversible (anergy) and can be broken by administration of the antigen in a highly organized fashion coupled to a foreign carrier (Bachmann and Zinkernagel, Ann. Rev. Immunol. 15:235-270 (1997)).

As indicated, methods for vaccinations against self-antigens derived from GnRH have recently been disclosed, e.g. in U.S. Pat. No. 5,897,863, or U.S. Pat. No. 6,132,720. The prior art anti-GnRH immunogens are, however, either not of sufficient potency to induce effective levels of anti-GnRH antibodies, or they need a specific linkers to positively affect the immune response (U.S. Pat. No. 6,132,720).

### BRIEF SUMMARY OF THE INVENTION

We have, now, found that the inventive compositions and vaccines, respectively, comprising GnRH peptides, fragments or variants thereof, coupled to VLPs were able to induce strong GnRH specific antibody responses, in particular without the need of specific immunogenic linkers or strong adjuvants. This indicates that GnRH peptides fragments or variants thereof, coupled to VLPs can be used to induce GnRH specific antibodies in humans and in animals, and thus resulting in reduced levels of gonadal steroids, gonad atrophy and infertility.

Furthermore, we have found that antibodies generated from vaccination with C- or N-terminally, preferably N-terminally linked GnRH peptides, fragments or variants thereof, of the invention to a VLP are able to bind GnRH. Therefore, GnRH peptides, fragments or variants thereof, coupled either C- or N-terminally, preferably N-terminally, to a virus-like particle (VLP), are capable of inducing highly specific anti-GnRH antibodies typically being capable of neutralizing the function of a GnRH before it continues to exert an unwanted effect in a disease or disorder related situation. Therefore, the present invention provides vaccination strategies against a disease or condition associated with GnRH, in particular as a treatment for boar taint, cancer, and other diseases where GnRH plays a role. The invention further provides vaccination strategies for the reduction of fertility of male and female animals using compositions of the invention.

The present invention, thus, provides a composition comprising (a) a virus like particle (VLP), and (b) at least one GnRH peptide or fragment or variant thereof, wherein (a) and (b) are linked with one another. Thus, the present invention provides for a composition comprising (a) a virus-like particle, and (b) at least one GnRH-peptide of the invention, wherein said GnRH-peptide of the invention is linked to said virus-like particle.

In a further preferred aspect, the present invention provides a composition comprising (a) a VLP with at least one first attachment site; and (b) at least one antigen or antigenic determinant with at least one second attachment site, wherein said antigen or antigenic determinant is a GnRH peptide of the invention, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said VLP interact through said association, preferably to form an ordered and repetitive antigen array. Preferred embodiments of VLPs suitable for use in the present invention are a virus-like particle of a RNA-phage or any other VLP having an inherent repetitive structure, preferably such a repetitive structure which is capable of forming an ordered and repetitive antigen array in accordance with the present invention. Very preferred embodiments of VLPs suitable for use in the present invention are a virus-like particle of a RNA-phage Qβ, a virus-like particle of a RNA-phage fr or a virus-like particle of a RNA-phage AP205.

The invention also provides a process for producing the VLPs of the invention. The VLPs and compositions of the invention are useful in the production of vaccines for the treatment of diseases or conditions associated with GnRH and as a pharmaceutical to prevent or cure such diseases, also to efficiently induce immune responses, in particular antibody responses. Furthermore, the compositions of the invention are particularly useful to efficiently induce self-specific immune responses within the indicated context.

In the present invention, a GnRH-peptide of the invention is bound to a VLP, preferably in an oriented manner, preferably yielding an ordered and repetitive GnRH-peptide antigen array. Furthermore, the highly repetitive and organized structure of the VLPs can mediate the display of the GnRH-peptide in a highly ordered and repetitive fashion leading to a highly organized and repetitive antigen array. Preferred arrays differ from prior art conjugates, in particular, in their highly organized structure, dimensions, in the repetitiveness of the antigen on the surface of the array, and in the efficacy. The latter is even the cases where no linker or short linkers are used.

In one aspect of the invention, the GnRH-peptide of the invention is expressed in a suitable expression host, or synthesized, while the VLP is expressed and purified from an expression host suitable for the folding and assembly of the VLP. GnRH-peptides of the invention may be chemically synthesized. The GnRH-peptide-array of the invention is then assembled by binding the GnRH-peptide of the invention to the VLP.

In a further aspect, the present invention provides a pharmaceutical composition comprising (a) a VLP, and (b) an acceptable pharmaceutical carrier. Typically and preferably, the present invention provides for a pharmaceutical composition, preferably a vaccine composition, comprising (a) a virus-like particle; and (b) at least one GnRH-peptide of the invention; and wherein said GnRH-peptide of the invention is linked to said virus-like particle.

In still a further aspect, the present invention provides for a method of producing a composition of the invention comprising (a) providing a virus-like particle; and (b) providing at least one GnRH-peptide of the invention; (c) combining said virus-like particle and said GnRH-peptide of the invention so that said GnRH-peptide is bound to said virus-like particle, in particular under conditions suitable for mediating a link between the VLP and the GnRH-peptide.

Analogously, the present invention provides a method of producing a VLP of the invention comprising: (a) providing a VLP with at least one first attachment site; (b) providing at least one GnRH-peptide of the invention with at least one added attachment site (furtheron called “second attachment site”), wherein said second attachment site being selected from the group consisting of (i) an attachment site not naturally occurring with said GnRH-peptide of the invention; and (ii) an attachment site naturally occurring within said GnRH-peptide of the invention; and wherein said second attachment site is capable of association to said first attachment site; and (c) combining said VLP and said at least one GnRH-peptide of the invention, wherein said GnRH-peptide of the invention and said VLP interact through said association, preferably to form an ordered and repetitive antigen array.

In another aspect, the present invention provides for a method of immunization comprising administering the composition or vaccine, respectively, of the invention to an animal, preferably a bird such as turkey, a mammal or a human.

In a further aspect, the present invention provides for a use of the composition or vaccine, respectively, of the invention for the manufacture of a medicament for treatment of GnRH related diseases.

In a still further aspect, the present invention provides for a use of the composition or vaccine of the invention for the preparation of a medicament for the therapeutic or prophylactic treatment of GnRH-related diseases. Furthermore, in a still further aspect, the present invention provides for a use of a composition or vaccine, respectively, of the invention, either in isolation or in combination with other agents for the manufacture of a composition, vaccine, drug or medicament for the treatment, therapy or prophylaxis of a disease or condition in an animal associated with GnRH, wherein the animal can be male or female. Said GnRH associated disease or condition can be any phenotype which is affected by gonadal steroid hormones, preferably fertility, gonadal steroid hormone dependent cancer, prostate cancer, boar taint in pork, beef and sheep, meat quality of male animals kept for meat production, gonadal steroid hormone related behaviour in male or female animals, for example aggression or sexual activity, and reproduction in wild life animals, modulation of thymus function and T-lymphocyte production in lymphocyte depleted individuals. In a preferred embodiment, the condition treated is the meat quality of male animals kept for meat production, preferably in rams, boars or bulls, very preferably in boars.

Therefore, the invention provides, in particular, vaccine compositions which are suitable for preventing and/or reducing or curing GnRH associated diseases or conditions related thereto, in particular gonadal steroid hormone dependent cancer, prostate cancer. The invention further provides immunization and vaccination methods, respectively, for treating or modulating conditions, in particular fertility, boar taint in pork, beef or sheep, increasing the meat quality of male animals kept for meat production particularly rams, boars, or bulls, gonadal steroid hormone related behaviour in animals, for example aggression or sexual activity, and reproduction in wild life animals. The inventive compositions may be used prophylactically or therapeutically.

A specific embodiment of the invention is a method of treating a GnRH associated disease or condition comprising administering the composition, the vaccine composition or the pharmaceutical composition of the invention to an animal or human, preferably to an animal.

More specifically, the invention provides for such method, wherein said disease or condition is selected from the group consisting of fertility, gonadal steroid hormone dependent cancer, prostate cancer, boar taint in pork, beef or sheep, meat quality of male animals kept for meat production, gonadal steroid hormone related behaviour in animals, and reproduction in wild life animals, wherein preferably said animals kept for meat production are rams, boars, or bulls.

In a preferred embodiment said disease or condition is fertility.

In another preferred embodiment the administering of the composition, the vaccine composition or the pharmaceutical composition of the invention is effected in an animal, preferably a bird or a mammal, more preferably a pet such as a dog, a cat or a rodent or a horse.

In another preferred embodiment the administering of the composition, the vaccine composition or the pharmaceutical composition of the invention is effected in an animal, preferably a bird or a mammal, more preferably a pet such as a dog, a cat or a rodent or a horse, wherein the animal is a female.

In another preferred embodiment the administering is effected by at most a first administration, a second and a third administration of said composition, said vaccine composition, or said pharmaceutical composition.

In another preferred embodiment the administering is effected by at most a first administration and a second administration of said composition, said vaccine composition, or said pharmaceutical composition.

In another preferred embodiment the administering is effected by only a first single administration of said composition, said vaccine composition, or said pharmaceutical composition.

In another specific embodiment the invention provides a method of treating a GnRH associated disease or condition comprising administering the composition, the vaccine composition or the pharmaceutical composition of the invention to an animal, wherein said animal is a pig, cattle or sheep, preferably a pig, and wherein said condition is boar taint in pork, beef or sheep, preferably pork. In more specific embodiment said administering is effected by at most a first administration, a second and a third administration of said composition, said vaccine composition, or said pharmaceutical composition. In another specific embodiment said administering is effected by at most a first administration and a second administration of said composition, said vaccine composition, or said pharmaceutical composition. In another specific embodiment said administering is effected by only a first single administration of said composition, said vaccine composition, or said pharmaceutical composition. In a preferred embodiment said first administration is effected 4 to 8 weeks prior to the slaughter of said pig, cattle or sheep.

The invention further provides a method of reducing boar taint in meat comprising administering the composition, the vaccine composition or the pharmaceutical composition of the invention to an animal, preferably a male animal, wherein said animal is a pig, cattle or sheep, preferably a pig, most preferably a male pig. In a preferred embodiment said administering is effected by at most a first administration, a second and a third administration of said composition, said vaccine composition, or said pharmaceutical composition. In another preferred embodiment said administering is effected by at most a first administration and a second administration of said composition, said vaccine composition, or said pharmaceutical composition. In another preferred embodiment said administering is effected by only a first single administration of said composition, said vaccine composition, or said pharmaceutical composition. In a specifically preferred embodiment said first administration is effected 4 to 8 weeks before slaughter of said pig, cattle or sheep.

For the reduction of boar taint male pig, cattle or sheep may be immunized at any developmental stage, preferably 4 to 8 weeks before slaughter. In a preferred embodiment, male pigs, cattle or sheep are immunized a first time between week 9 and week 18 and a second time 4 to 8 weeks before slaughter. In a further preferred embodiment male pigs, cattle or sheep are immunized only once, preferably between week 16 and 20. Alternatively, male pigs, cattle or sheep are immunized only once 4 to 8 weeks before slaughter.

The invention further provides a method of preventing, reducing or eliminating the fertility of an animal comprising administering the composition, the vaccine composition or the pharmaceutical composition of the invention to said animal, wherein said animal preferably is a female. In a preferred embodiment said animal is a mammal, preferably a pet such as a dog, cat or a rodent, or a horse. In a preferred embodiment said administering is effected by at most a first administration, a second and a third administration of said composition, said vaccine composition, or said pharmaceutical composition. In another preferred embodiment said administering is effected by at most a first administration and a second administration of said composition, said vaccine composition, or said pharmaceutical composition. In another preferred embodiment said administering is effected by only a first single administration of said composition, said vaccine composition, or said pharmaceutical composition. In a specifically preferred embodiment the fertility of said animal is prevented, reduced or eliminated permanently, meaning that the animal does not gain or regain fertility throughout its lifespan.

The vaccine or composition used for immunisation and administration, respectively, can be administered to the animal by any mode that is medically acceptable, meaning any mode that produces effective levels of the active compounds without causing clinically unacceptable adverse effects. Such modes of administration include oral, rectal, parenteral, intracisternal, intravaginal, intraperitoneal, topical (as by powders, ointments, drops or transdermal patch), bucal, or as an oral or nasal spray. The term “parenteral” as used herein refers to modes of administration which include intravenous, intramuscular, intraperitoneal, intrasternal, subcutaneous and intraarticular injection and infusion. The composition of the invention can also be injected directly in a lymph node. Preferably the vaccines and compositions of the invention are administered by subcutaneous injection.

The vaccination of an animal or the administration of the inventive compositions to an animal, respectively, for the purposes of the invention typically require 10 to 2000 μg, preferably 100 to 1500 μg, more preferably 400 to 1000 μg and most preferably about 400 μg of the vaccine or the composition of the invention per immunization for animals such as pig, sheep or cattle and 10 to 200 μg per immunization for companion animals. In a preferred embodiment 100 to 1000 μg, preferably about 400 μg of the vaccine or the composition of the invention are administered per immunization to male pig in a prime and boost immunization strategy for the reduction of boar taint. In a further preferred embodiment about 800 to 2000 μg, preferably 1000 to 1500 μg and most preferable about 1000 μg of the vaccine or composition of the invention are administered to male pig, cattle or sheep in a single shot immunization strategy for the reduction of boar taint.

As would be understood by one of ordinary skill in the art, when compositions of the invention are administered to an animal or a human, they may be in a composition which contains salts, buffers, adjuvants, or other substances which are desirable for improving the efficacy of the composition. Examples of materials suitable for use in preparing pharmaceutical compositions are provided in numerous sources including Remington's Pharmaceutical Sciences (Osol, A, ed., Mack Publishing Co. (1990)). In a preferred embodiment the compositions of the invention comprise or are administered with an adjuvant, preferably DEAE Dextran, wherein the administration of the compositions of the invention and the administration of the adjuvant can be effected simultaneously or one after the other, in any temporal order and, preferrably, with a time interval which is not longer than one week. Preferably, the time interval is one day or less, most preferably the inventive composition and the adjuvant are administered simultaneously, most preferably the inventive composition and the adjuvant are mixed with each other. DEAE Dextran is particularly suitable to enhance the immune response of animals, preferably pigs, to the vaccines and the compositions of the invention.

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention may be administered by various methods known in the art, but will normally be administered by injection, infusion, inhalation, oral administration or other suitable physical methods. The compositions may alternatively be administered intramuscularly, intravenously, or subcutaneously. Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Other embodiments of the present invention will be apparent to one of ordinary skill in light of what is known in the art, the following description of the invention, and the claims.

### BRIEF DESCRIPTION OF THE FIGURES

FIG. 1. Qβ VLPs were derivatized and coupled with (A) peptides CGG-GnRH and GnRH-GGC and with (B) peptides C-GnRH and GnRH-C. FIG. 1(A) shows: M: 7708S protein marker (NE Biolabs); lane 1: 10 μg Qβ; lane 2: 10 μg derivatized Qβ; lane 3: 10 μg Qβ-CGG-GnRH; lane 4: 10 μg GnRH-GGC-Qβ; FIG. 1(B) shows: M: 7708S protein marker (NE Biolabs); lane 1: 10 μg Qβ; lane 2: 10 μg derivatized Qβ; lane 3: 10 μg Qβ-C-GnRH (6); lane 4: 10 μg GnRH-C-Qβ.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

### 1. DEFINITIONS

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. A variety of adjuvants can be used. Examples include complete and incomplete Freund's adjuvant, aluminum hydroxide and modified muramyldipeptide. Further adjuvants are mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants can also comprise a mixture of these substances. For the purposes of the invention a specifically preferred adjuvant is DEAE Dextran.

Immunologically active saponin fractions having adjuvant activity derived from the bark of the South American tree Quillaja Saponaria Molina are known in the art. For example QS21, also known as QA21, is an Hplc purified fraction from the Quillaja Saponaria Molina tree and it's method of its production is disclosed (as QA21) in U.S. Pat. No. 5,057,540. Quillaja saponin has also been disclosed as an adjuvant by Scott et al., Int. Archs. Allergy Appl. Immun., 1985, 77, 409. Monosphoryl lipid A and derivatives thereof are known in the art. A preferred derivative is 3 de-o-acylated monophosphoryl lipid A, and is known from British Patent No. 2220211. Further preferred adjuvants are described in WO 00/00462, the disclosure of which is herein incorporated by reference.

However, an advantageous feature of the present invention is the high immunogenicty of the modified VLPs of the invention, even in the absence of adjuvants. As already outlined herein or will become apparent as this specification proceeds, vaccines and pharmaceutical compositions devoid of adjuvants are provided, in further alternative or preferred embodiments, leading to vaccines and pharmaceutical compositions for treating GnRH-related diseases while being devoid of adjuvants and, thus, having a superior safety profile since adjuvants may cause side-effects. The term “devoid” as used herein in the context of vaccines and pharmaceutical compositions for treating GnRH-related diseases refers to vaccines and pharmaceutical compositions that are used essentially without adjuvants, preferably without detectable amounts of adjuvants.

Amino acid linker: An “amino acid linker”, or also just termed “linker” within this specification, as used herein, either associates the GnRH-peptide of the invention with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occurring amino acids or unnatural amino acids known in the art, all-L or all-D or mixtures thereof. However, an amino acid linker comprising a molecule with a sulfhydryl group or cysteine residue is also encompassed within the invention. Such a molecule comprises preferably a C1-C6 alkyl-, cycloalkyl (C5, C6), aryl or heteroaryl moiety. However, in addition to an amino acid linker, a linker comprising preferably a C1-C6 alkyl-, cycloalkyl-(C5, C6), aryl- or heteroaryl-moiety and devoid of any amino acid(s) shall also be encompassed within the scope of the invention. Association between the GnRH-peptide of the invention or optionally the second attachment site and the amino acid linker is preferably by way of at least one covalent bond, more preferably by way of at least one peptide bond.

As used herein, the term “linker which does not essentially affect the immune response against GnRH” refers to a linker that does not induce a significant antibody titer against itself and does not make a critical contribution to or significantly influence the immune response against GnRH. Thus, the vaccines and compositions of the invention using GnRH and said linker, typically and preferably induce no significant immune response against the linker or against the linker plus GnRH. Such a linker is typically three or less than three amino acids in length. Methods to determine whether a linker induces an antibody response are well known in the art, e.g. one method involves testing sera of immunized animals in an ELISA such as the ELISA method described in Example 2.

Animal: As used herein, the term “animal” is meant to include, for example, humans, sheep, elks, deer, mule minks, monkeys, horses, bulls, cattle, pigs, goats, dogs, cats, rats, and mice. Preferred animals are mammals, more preferred animals are eutherians, and even more preferred animals are vertebrates.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies can be from any animal origin including birds and mammals. Preferably, the antibodies are human, murine, rabbit, goat, rat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T-cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-cell epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. Preferred antigens, and thus preferred GnRH-peptides, are short peptides (5-10 aa residues, or 6-8 aa residues, respectively) which do not result in a T-cell response (B-cell epitopes only).

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar, or any combination thereof, preferably the nature of the association is covalent.

Attachment Site, First: As used herein, the phrase “first attachment site” refers to an element of non-natural or natural origin, to which the second attachment site located on the GnRH-peptide of the invention may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. The first attachment site is located, typically and preferably on the surface, of the virus-like particle. Multiple first attachment sites are present on the surface of the core and virus-like particle, respectively, typically in a repetitive configuration.

Attachment Site, Second: As used herein, the phrase “second attachment site” refers to an element associated with the GnRH-peptide of the invention to which the first attachment site located on the surface of the virus-like particle may associate. It refers to an element which is naturally occurring with or which is artificially added to the GnRH peptide of the invention and to which the first attachment site may be linked The second attachment site of the GnRH-peptide may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is the sulfhydryl group, preferably of an amino acid cysteine. In certain embodiments of the invention at least one second attachment site may be added to the GnRH-peptide of the invention. The term “GnRH-peptide of the invention with at least one second attachment site” refers, therefore, to a GnRH-peptide of the invention comprising at least the GnRH-peptide of the invention and a second attachment site. However, in particular for a second attachment site, which is of non-natural origin, i.e. not naturally occurring within the GnRH-peptide of the invention, such a construct typically and preferably further comprises a “linker”. In another preferred embodiment the second attachment site is associated with the GnRH peptide of the invention through at least one covalent bond, preferably through at least one peptide bond. In a further embodiment, the second attachment site is naturally occurring within the GnRH peptide of the invention. In yet another preferred embodiment, the second attachment site is artificially added to the GnRH peptide of the invention through an amino acid linker, preferably comprising a cysteine, by protein fusion.

Bound: As used herein, the term “bound” as well as the term “linked”, which is herein used equivalently, refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds such as thioether, carbon-phosphorus bonds, and the like. The terms “bound” and “linked” are broader than and include terms such as “coupled,” “fused” and “attached”, which terms are preferred interpretations of the terms “bound” and “linked. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only encompass a direct linkage of the at least one GnRH-peptide and the virus-like particle but also, alternatively and preferably, an indirect linkage of the at least one GnRH-peptide and the virus-like particle through intermediate molecule(s), and hereby typically and preferably by using at least one, preferably one, heterobifunctional cross-linker. Moreover, the term “linked” as used herein shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “Qβ CP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly Qβ CP, with a minor content of A1 protein.

Coupled: As used herein, the term “coupled” refers to attachment by covalent bonds or by strong non-covalent interactions, typically and preferably to attachment by covalent bonds. Any method normally used by those skilled in the art for the coupling of biologically active materials can be used in the present invention.

Effective Amount As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount”.

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 4 such amino acids, and more usually, consists of at least about 4-10 such amino acids. If the epitope is an organic molecule, it may be as small as Nitrophenyl. Preferred epitopes are the GnRH-peptides of the invention, which are believed to be B-type epitopes.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the term “GnRH-peptide” or “GnRH peptide of the invention” is a peptide comprising, or alternatively essentially consisting of, or alternatively consisting of at least one, preferably one, mammalian GnRH, and hereby in particular at least one amino acid sequence, preferably one amino acid sequence of SEQ ID NO: 1 or SEQ ID NO: 28, preferably of SEQ ID NO: 1, or fragments or variants thereof. In some embodiments, the GnRH peptide comprises or contains N-terminal puroglutamic acid (pGlu or pE). In other embodiments, the GnRH peptide comprises or contains C-terminal glycine amide (G-NH2). Typically and preferably, the GnRH peptide comprises or contains C-terminal glycine amide (G-NH2) if the amino acid linker or second attachment site, respectively, of the invention is associated with the N-terminus of the GnRH peptide. Preferred GnRH peptides comprising C-terminal glycine amide are selected from the group consisting of, without limitation, peptides with the amino acid sequence of SEQ ID NOs: 1, 6-9, 28, and 43. In another preferred embodiment, the GnRH peptide comprises or contains N-terminal puroglutamic acid (pE) if the amino acid linker or second attachment site, respectively, of the invention is associated with the C-terminus of the GnRH peptide. Preferred GnRH peptides comprising N-terminal puroglutamic acid are selected from the group consisting of, without limitation, peptides with the amino acid sequence of SEQ ID NO: 1 and SEQ ID NOs: 28-36. In another embodiment of the invention, the GnRH peptide comprises or contains more than one GnRH peptide or fragment thereof, for example two (e.g. SEQ ID NOs: 34, 35, or 36), three or more GnRH peptides or fragments thereof in tandem. The tandem-GnRH peptide of the invention also comprises peptides in which the GnRH sequences are interconnected via spacer. The nature of the spacer group may greatly vary from one or more amino acids to a shorter or longer hydrocarbon chain and other compound groups or molecules.

As indicated, the term “GnRH peptide” or “GnRH peptide of the invention”, as defined herein, should also refer to fragments of mammalian GnRH, and hereby in particular of SEQ ID NO: 1 or SEQ ID NO: 28, preferably of SEQ ID NO: 1. Typically and preferably, the term “GnRH peptide” or “GnRH peptide of the invention”, as defined herein, refers to fragments of mammalian GnRH, and hereby in particular of SEQ ID NO: 1 or SEQ ID NO: 28, preferably of SEQ ID NO: 1, wherein said fragments comprise or alternatively consist of at least 4, 5, 6, 7, 8, 9, or 10 contiguous amino acids of a GnRH peptide as defined herein as well as any polypeptide having equal or more than 60%, preferably equal or more than 70%, more preferably equal or more than 80% and even more preferably equal or more than 90% amino acid sequence identity thereto. Preferred GnRH peptides and GnRH fragments, respectively, comprise or consist of amino acid residues 2 to 10 (SEQ ID NO: 6), 3 to 10 (SEQ ID NO: 7), 4 to 10 (SEQ ID NO: 8), 5 to 10 (SEQ ID NO: 9), 6 to 10 (SEQ ID NO: 43), 1 to 9 (SEQ ID NO: 29), 1 to 8 (SEQ ID NO: 30), 1 to 7 (SEQ ID NO: 31), 1 to 6 (SEQ ID NO: 32) or 1 to 5 (SEQ ID NO: 33) of the GnRH peptide sequence.

As indicated, the term “GnRH peptide” or “GnRH peptide of the invention”, as defined herein, should also refer to variants of mammalian GnRH, and hereby in particular to variants of SEQ ID NO: 1 or SEQ ID NO: 28, preferably of SEQ ID NO: 1. Typically and preferably, the term “variant” refers to a polynucleotide or polypeptide or peptide that differs from the GnRH polynucleotide or polypeptide, but retains the essential properties thereof. A typical and preferred variant of a GnRH peptide differs in amino acid sequence from mammalian GnRH, and hereby in particular from SEQ ID NO: 1 or SEQ ID NO: 28, preferably from SEQ ID NO: 1. Generally, alterations are limited so that the sequences of mammalian GnRH and the variant are closely similar overall and, in many regions, identical. Typically and preferably, the term “GnRH peptide” or “GnRH peptide of the invention”, as defined herein, should also refer to variants of mammalian GnRH, and hereby in particular to variants of SEQ ID NO: 1 or SEQ ID NO: 28, preferably of SEQ ID NO: 1, wherein said variants differ in amino acid sequence by one or more, preferably at most three, more preferably one or two, even more preferably one substitutions, preferably conservative substitutions, insertions or deletions, and/or wherein said variants are peptides having one or more, preferably at most three, more preferably one or two, even more preferably one post-translational modifications, for instance glycosylation, phosphorylation, methylation, ADIP ribosylation and the like, and/or wherein said variants comprise, or consists of, any polypeptide comprising, or alternatively or preferably consisting of, any natural or genetically engineered polypeptide having equal or more than 60%, preferably equal or more than 70%, more preferably equal or more than 80% and even more preferably equal or more than 90% amino acid sequence identity with the mammalian GnRH peptide, and hereby in particular of SEQ ID NO: 1 or SEQ ID NO: 28, preferably of SEQ ID NO: 1.

A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. Embodiments of the post-translational modifications include methylation of the N-terminal amino acid, phosphorylations of serines and threonines and modification of C-terminal glycines. Guidance in determining which amino acid residues may be substituted, inserted, or deleted without abolishing biological or immunological activity may be found using computer programs well known in the art, for example, LASERGENE software (DNASTAR). A preferred variant of a polynucleotide or peptide may be naturally occurring such as an allele, or it may be a variant that is not known to occur naturally. Non-naturally occurring variants of polynucleotides and peptides may be made by mutagenesis techniques or by direct synthesis.

Preferred embodiments of GnRH variants are truncation, internal deletion, or substitution forms of GnRH peptides. Preferred GnRH variants comprise a peptide with a substitution of the sixth amino acid Gly of the GnRH 1-10 peptide (SEQ ID NO: 1) by a Lys (resulting in SEQ ID NO: 42). Preferably, GnRH fragments and variants are capable of inducing the production of antibody in vivo, which specifically binds to GnRH as verified by, for example ELISA, by incubating GnRH with sera taken from animal or human immunized with GnRH peptide.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

The GnRH-peptide may be obtained by recombinant expression in eukaryotic or prokaryotic expression systems as GnRH-peptide alone, but preferably as a fusion with other amino acids or proteins, e.g. to facilitate folding, expression or solubility of the GnRH-peptide or to facilitate purification of the GnRH-peptide. Preferred are fusions between GnRH-peptides and subunit proteins of VLPs or capsids. In such a case, one or more amino acids may be added N- or C-terminally to GnRH-peptides. In one embodiment, the GnRH-peptide is at the N-terminus of a fusion polypeptide, i.e. coupled or linked via its own C-terminus to its fusion partner.

Alternatively and preferably, to enable coupling of GnRH-peptides to subunit proteins of VLPs or capsids, at least one second attachment site may be added to the GnRH-peptide. Alternatively GnRH-peptides may be synthesized using methods known to the art, in particular by organic-chemical peptide synthesis. Such peptides may even contain amino acids which are not present in the mammalian GnRH peptide. The peptides may be modified by, e.g., phosphorylation, but this modification is not necessary for effective modified VLPs of the invention.

Residue: As used herein, the term “residue” is meant to mean a specific amino acid in a polypeptide backbone or side chain.

Immune response: As used herein, the term “immune response” refers to a humoral immune response and/or cellular immune response leading to the activation or proliferation of B- and/or T-lymphocytes and/or and antigen presenting cells.

In some instances, however, the immune responses may be of low intensity and become detectable only when using at least one substance in accordance with the invention. “Immunogenic” refers to an agent used to stimulate the immune system of a living organism, so that one or more functions of the immune system are increased, and directed towards the immunogenic agent. A substance which “enhances” an immune response refers to a substance in which an immune response is observed that is greater or intensified or deviated in any way with the addition of the substance when compared to the same immune response measured without the addition of the substance.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Immunosterilization/Immunocastration: A method for reducing gonadotropic hormone and, thus, gonadal steroid hormone production in male and female animals by immunologic means, thereby interfering with fertility and other gonadal steroid hormone related phenotypes, diseases, disorders, conditions and behaviour. In male animals the terms immunosterilization and immunocastration can be used interchangeably.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform spacial arrangement of the antigens or antigenic determinants with respect to the virus-like particle. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 1 to 30 nanometers, preferably 2 to 15 nanometers, even more preferably 2 to 10 nanometers, even again more preferably 2 to 8 nanometers, and further more preferably 3 to 7 nanometers.

Polypeptide: As used herein, the terms “polypeptide” and “peptide” refer to molecules composed of monomers (amino acids) linearly linked by amide bonds (also known as peptide bonds). They indicate a molecular chain of amino acids. Preferred peptides of the invention are pentapeptides, hexapeptides, heptapeptides, octapeptides nonapeptides, and decapeptides. For the purpose of this invention, a polypeptide is regarded as a peptide. These terms also refer to post-expression modifications of the polypeptide or peptide, for example, glycosylations, acetylations, phosphorylations, and the like. A recombinant or derived polypeptide or peptide is not necessarily translated from a designated nucleic acid sequence. It may also be generated in any manner, including chemical synthesis, which is preferred for peptides.

Self antigen: As used herein, the tem “self antigen” refers to proteins encoded by the host's DNA and products generated by proteins or RNA encoded by the host's DNA are defined as self. In addition, proteins that result from a combination of two or several self-molecules may also be considered self.

Treatment: As used herein, the terms “treatment”, “treat”, “treated” or “treating” refer to prophylaxis and/or therapy. When used with respect to a GnRH related disease or condition, for example, the term refers to a prophylactic treatment which increases the resistance of a subject to develop a GnRH associated disease or condition or, in other words, decreases the likelihood that the subject will develop an GnRH associated disease or condition or will show signs of illness attributable to an GnRH associated disease or condition, as well as a treatment after the subject has developed an GnRH associated disease or condition in order to fight the GnRH associated disease or condition, e.g., reduce or eliminate the GnRH associated disease or condition or prevent it from becoming worse.

Vaccine: As used herein, the term “vaccine” refers to a formulation which contains the modified VLP of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Typically, the modified VLP of the invention, preferably induces a predominant B-type response, more preferably a B-type response only, which can be a further advantage.

Optionally, the vaccine of the present invention additionally includes an adjuvant which can be present in either a minor or major proportion relative to the compound of the present invention.

Virus-like particle (VLP): As used herein, the term “virus-like particle” refers to a structure resembling a virus particle. Moreover, a virus-like particle in accordance with the invention is non-replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAgs have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein.” A specific example is the VLP of Qβ coat protein. In this particular case, the VLP of Qβ coat protein may either be assembled exclusively from Qβ CP subunits (generated by expression of a QβCP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

One, a, or an: When the terms “one,” “a,” or “an” are used in this disclosure, they mean “at least one” or “one or more,” unless otherwise indicated. Preferably, they mean “one”.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2nd edition, (1998)) and antibody-based technologies (Harlow, E. and Lane, D., Antibodies: A Laboratory Manual, Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., Protein Purification Principles and Practice, 3rd ed., Springer-Verlag, New York (1994)) are also adequately described in the literature, all of which are incorporated herein by reference.

### 2. COMPOSITIONS AND METHODS FOR ENHANCING AN IMMUNE RESPONSE

The disclosed invention provides compositions and methods for enhancing an immune response against a GnRH-peptide in an animal, preferably a human being. Compositions of the invention comprise, or alternatively consist of (a) a VLP; and (b) at least one GnRH-peptide, wherein a) and b) are linked with one another. Said GnRH-peptide consists of a peptide with a length of 5 to 10 amino acid residues. Preferred GnRH-peptides comprise, and more preferably consist of, the peptide QHWSYGLRPG (SEQ ID NO: 28) or more preferably of the peptide EHWSYGLRPG (SEQ ID NO: 1) or fragments or variant thereof (SEQ ID NOs: 2-9 and 28-36, and 42-43). In a particularly preferred embodiment, the GnRH peptide of the invention comprises or more preferably consists of SEQ ID NO: 1.

In a preferred embodiment the GnRH-peptide of the invention is bound to the virus-like particle so as to form an ordered and repetitive antigen-VLP-array. In a further preferred embodiment the GnRH-peptide comprises or typically and preferably consists of a peptide with a length of 5 to 10 amino acid residues. Alternatively, the lower limit in the above-mentioned length range can preferably be 5, 6, 7, 8 or 9 amino acid residues.

Virus-like particles in the context of the present application refer to VLPs that are described in detail in WO 03/024481 on page 39 to 59, the disclosure of which is incorporated herein by reference. Examples of VLPs include, but are not limited to, the capsid proteins of Hepatitis B virus, measles virus, Sindbis virus, rotavirus, foot-and-mouth-disease virus, Norwalk virus, the retroviral GAG protein, the retrotransposon Ty protein p1, the surface protein of Hepatitis B virus, human papilloma virus, Ty and preferably RNA phages such as fr-phage, GA-phage, AP205-phage, and, in particular, Qβ-phage. In a more specific embodiment, the VLP can comprise, or alternatively essentially consist of, or alternatively consist of recombinant polypeptides, or fragments thereof. The virus-like particle can further comprise, or alternatively essentially consist of, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts.

In a preferred embodiment, the virus-like particle comprises, preferably consists essentially of, or alternatively consists of recombinant proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7, and m) bacteriophage AP205.

In a further preferred embodiment of the present invention, the recombinant proteins comprise, or alternatively consist essentially of, or alternatively consist of coat proteins of RNA phages.

Specific preferred examples of bacteriophage coat proteins which can be used to prepare compositions of the invention are described in detail in WO 03/024481 (page 41 last paragraph to page 49 second paragraph), the disclosure of which is incorporated herein by reference, and which include the coat proteins of RNA bacteriophages such as bacteriophage Qβ (SEQ ID NO: 10; PIR Database, Accession No. VCBPQβ referring to Qβ CP and SEQ ID NO: 11, Accession No. AAA16663 referring to Qβ A1 protein), bacteriophage R17 (SEQ ID NO: 12, PIR Accession No. VCBPR7), bacteriophage fr (SEQ ID NO: 13, PIR Accession No. VCBPFR), bacteriophage GA (SEQ ID NO: 14; GenBank Accession No. NP-040754), bacteriophage SP (SEQ ID NO: 15; GenBank Accession No. CAA30374 referring to SP CP and SEQ ID NO: 16; Accession No. NP_{—}695026 referring to SP A1 protein), bacteriophage MS2 (SEQ ID NO: 17; PIR Accession No. VCBPM2), bacteriophage M11 (SEQ ID NO: 18; GenBank Accession No. AAC06250), bacteriophage MX1 (SEQ ID NO: 19; GenBank Accession No. AAC14699), bacteriophage NL95 (SEQ ID NO: 20; GenBank Accession No. AAC14704), bacteriophage f2 (SEQ ID NO: 21; GenBank Accession No. P03611), bacteriophage PP7 (SEQ ID NO: 22), and bacteriophage AP205 (SEQ ID NO: 39). Furthermore, the A1 protein of bacteriophage Qβ or C-terminal truncated forms missing as much as 100, 150 or 180 amino acids from its C-terminus may be incorporated in a capsid assembly of Qβ coat proteins. Generally, the percentage of QβA1 protein relative to Qβ CP in the capsid assembly will be limited, in order to ensure capsid formation.

Further preferred virus-like particles of RNA-phages, in particular of Qβ in accordance of this invention are disclosed in WO 02/056905, the disclosure of which is herewith incorporated by reference in its entirety. In particular, a detailed description of the preparation of VLP particles from Qβ is disclosed in Example 18 of WO 02/056905.

In a further preferred embodiment of the present invention, the virus-like particle comprises, or alternatively consists essentially of, or alternatively consists of recombinant proteins, or fragments thereof, of a RNA-phage, wherein the recombinant proteins comprise, alternatively consist essentially of or alternatively consist of mutant coat proteins of a RNA phage, preferably of mutant coat proteins of the RNA phages mentioned above. In one embodiment, the mutant coat proteins are fusion proteins with a GnRH-peptide of the invention. In another preferred embodiment, the mutant coat proteins of the RNA phage have been modified by removal of at least one, or alternatively at least two, lysine residue by way of substitution, or by addition of at least one lysine residue by way of substitution; alternatively, the mutant coat proteins of the RNA phage have been modified by deletion of at least one, or alternatively at least two, lysine residue, or by addition of at least one lysine residue by way of insertion. The deletion, substitution or addition of at least one lysine residue allows varying the degree of coupling, i.e. the amount of GnRH peptides per subunits of the VLP of the RNA-phages, in particular, to match and tailor the requirements of the vaccine.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLPs can, thus, be used in the practice of the invention: “Qβ-240” (Lys13-Arg; SEQ ID NO: 23), “Qβ-243” (Asn 10-Lys; SEQ ID NO: 24), “Qβ-250” (Lys 2-Arg, Lys13-Arg; SEQ ID NO: 25), “Qβ-251” (SEQ ID NO: 26) and “Qβ-259” (Lys 2-Arg, Lys16-Arg; SEQ ID NO: 27). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO: 23; b) the amino acid sequence of SEQ ID NO: 24; c) the amino acid sequence of SEQ ID NO: 25; d) the amino acid sequence of SEQ ID NO: 26; and e) the amino acid sequence of SEQ ID NO: 27. The construction, expression and purification of the above indicated Qβ coat proteins, mutant Qβ coat protein VLPs and capsids, respectively, are described in WO 02/056905. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading frames not present in related phages; a lysis gene and an open reading frame playing a role in the translation of the maturation gene (Klovins, J., et al., J. Gen. Virol. 83:1523-33 (2002)). WO 2004/007538 describes, in particular in Example 1 and Example 2, how to obtain VLP comprising AP205 coat proteins, and hereby in particular the expression and the purification thereto. WO 2004/007538, and hereby in particular the indicated Examples, are incorporated herein by way of reference. AP205 VLPs are highly immunogenic, and can be linked with GnRH peptides of the invention to generate vaccine constructs displaying the GnRH peptides of the invention oriented in a repetitive manner. High titers are elicited against the so displayed GnRH peptides of the invention showing that bound GnRH peptides of the invention are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the substitution of proline at amino acid 5 to threonine may also be used in the practice of the invention and leads to further preferred embodiments of the invention. The cloning of the AP205Pro-5-Thr and the purification of the VLPs are disclosed in WO 2004/007538, and therein, in particular within Example 1 and Example 2. The disclosure of WO 2004/007538, and, in particular, Example 1 and Example 2 thereof is explicitly incorporated herein by way of reference.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of a GnRH-peptide of the invention to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The invention further includes compositions and vaccine compositions, respectively, comprising proteins, which comprise, or alternatively consist essentially of, or alternatively consist of amino acid sequences which are at least 80%, 85%, 90%, 95%, 97%, or 99% identical to wild-type proteins which form ordered arrays and having an inherent repetitive structure, respectively.

In other embodiments, the invention further includes compositions comprising proteins, which comprise, or alternatively consist essentially of, or alternatively consist of amino acid sequences which are at least 80%, 85%, 90%, 95%, 97%, or 99% identical to any of the amino acid sequences of above mentioned bacteriophages.

Proteins suitable for use in the present invention also include C-terminal truncation mutants of proteins which form capsids or capsid-like structures, or VLPs. Specific examples of such truncation mutants include proteins having an amino acid sequence of above mentioned bacteriophages and of the sequences shown in any of SEQ ID NOs: 10-15 where 1, 2, 5, 7, 9, 10, 12, 14, 15, or 17 amino acids have been removed from the C-terminus. Typically, theses C-terminal truncation mutants will retain the ability to form capsids or capsid-like structures.

Further proteins suitable for use in the present invention also include N-terminal truncation mutants of proteins which form capsids or capsid-like structures. Specific examples of such truncation mutants include proteins having an amino acid sequence of above mentioned bacteriophages and of the sequences shown in any of SEQ ID NOs: 10-15 where 1, 2, 5, 7, 9, 10, 12, 14, 15, or 17 amino acids have been removed from the N-terminus. Typically, these N-terminal truncation mutants will retain the ability to form capsids or capsid-like structures.

In a preferred embodiment, the mutant coat proteins of said RNA phage have been modified by removal, or by addition of at least one lysine residue by way of substitution. In another preferred embodiment, the mutant coat proteins of said RNA phage have been modified by deletion of at least one lysine residue or by addition of at least one lysine residue by way of insertion. In a preferred embodiment, the virus-like particle comprises recombinant proteins or fragments thereof, of RNA-phage Q [symbol] or alternatively of RNA-phage fr, or of RNA-phage AP205.

As previously stated, the invention includes virus-like particles or recombinant forms thereof. In one further preferred embodiment, the particles used in compositions of the invention are composed of a Hepatitis B core protein (HBcAg) or a fragment of a HBcAg. In a further embodiment, the particles used in compositions of the invention are composed of a Hepatitis B core protein (HBcAg) or a fragment of a HBcAg protein, which has been modified to either eliminate or reduce the number of free cysteine residues. Zhou et al. (J. Virol. 66:5393 5398 (1992)) demonstrated that HBcAgs which have been modified to remove the naturally resident cysteine residues retain the ability to associate and form capsids. Thus, VLPs suitable for use in compositions of the invention include those comprising modified HBcAgs, or fragments thereof, in which one or more of the naturally resident cysteine residues have been either deleted or substituted with another amino acid residue (e.g., a serine residue).

The HBcAg is a protein generated by the processing of a Hepatitis B core antigen precursor protein. A number of isotypes of the HBcAg have been identified and their amino acids sequences are readily available to those skilled in the art. In most instances, compositions and vaccine compositions, respectively, of the invention will be prepared using the processed form of a HBcAg (i.e., an HBcAg from which the N-terminal leader sequence of the Hepatitis B core antigen precursor protein has been removed).

Further, when HBcAgs are produced under conditions where processing will not occur, the HBcAgs will generally be expressed in “processed” form. For example, when an E. coli expression system directing expression of the protein to the cytoplasm is used to produce HBcAgs of the invention, these proteins will generally be expressed such that the N-terminal leader sequence of the Hepatitis B core antigen precursor protein is not present.

Specific preferred examples of HBcAg proteins, such as for example the HBcAg of SEQ ID NO: 37 or variants thereof, which can be used to prepare compositions of the invention are described in detail in WO 03/024481 (page 52 fourth paragraph to page 58 last paragraph), the disclosure of which is incorporated herein by reference.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in WO 02/056905. For the latter application, it is in particular referred to Example 23, 24, 31 and 51. All three documents are explicitly incorporated herein by reference.

A number of naturally occurring HBcAg variants suitable for use in the practice of the present invention has been identified (e.g. Yuan et al., (J. Virol. 73:10122-10128 (1999)). Further HBcAg variants that are suitable for use in the practice of the present invention are disclosed in WO 03/024481 (page 54 third paragraph to page 55 first paragraph) the disclosure of which is incorporated herein by reference. Further HBcAg variants suitable for use in the compositions of the invention, and which may be further modified according to the disclosure of this specification are described in WO 00/198333, WO 00/177158 and WO 00/214478.

In a further preferred embodiment, the virus-like particle comprises, or alternatively consists essentially of, or alternatively consists of recombinant proteins of SEQ ID NO: 37.

Whether the amino acid sequence of a polypeptide has an amino acid sequence that is at least 80%, 85%, 90%, 95%, 97% or 99% identical to one of the above amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The amino acid sequences of the hereinabove mentioned HBcAg variants and precursors are relatively similar to each other. Thus, reference to an amino acid residue of a HBcAg variant located at a position which corresponds to a particular position in SEQ ID NO: 37, refers to the amino acid residue which is present at that position in the amino acid sequence shown in SEQ ID NO: 37. The homology between these HBcAg variants is for the most part high enough among Hepatitis B viruses that infect mammals so that one skilled in the art would have little difficulty reviewing both the amino acid sequence shown in SEQ ID NO: 37 and that of a particular HBcAg variant and identifying “corresponding” amino acid residues.

The invention also includes vaccine compositions which comprise HBcAg variants of Hepatitis B viruses which infect birds, as wells as vaccine compositions which comprise fragments of these HBcAg variants. For these HBcAg variants one, two, three or more of the cysteine residues naturally present in these polypeptides could be either substituted with another amino acid residue or deleted prior to their inclusion in vaccine compositions of the invention.

As discussed above, the elimination of free cysteine residues reduces the number of sites where toxic components can bind to the HBcAg, and also eliminates sites where cross-linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue.

In other embodiments, compositions and vaccine compositions, respectively, of the invention will contain HBcAgs from which the C-terminal region (e.g., amino acid residues 145-185 or 150-185 of SEQ ID NO: 37) has been removed. Thus, additional modified HBcAgs suitable for use in the practice of the present invention include C-terminal truncation mutants. Suitable truncation mutants include HBcAgs where 1, 5, 10, 15, 20, 25, 30, 34, 35, amino acids have been removed from the C-terminus.

HBcAgs suitable for use in the practice of the present invention also include N-terminal truncation mutants; Suitable truncation mutants include modified HBcAgs where 1, 2, 5, 7, 9, 10, 12, 14, 15, or 17 amino acids have been removed from the N-terminus.

Further HBcAgs suitable for use in the practice of the present invention include N- and C-terminal truncation mutants. Suitable truncation mutants include EBcAgs where 1, 2, 5, 7, 9, 10, 12, 14, 15, and 17 amino acids have been removed from the N-terminus and 1, 5, 10, 15, 20, 25, 30, 34, 35 amino acids have been removed from the C-terminus.

The invention further includes compositions and vaccine compositions, respectively, comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of GnRH-peptide of the invention to the VLP of HBcAg. In preferred embodiments, modified VLPs of the invention, and compositions of the invention are prepared using a HBcAg comprising, or alternatively consisting of, amino acids 1-144, or 1-149, 1-185 of SEQ ID NO: 37, which is modified so that the amino acids corresponding to positions 79 and 80 are replaced with a peptide having the amino acid sequence of Gly-Gly-Lys-Gly-Gly (SEQ ID NO: 40) resulting in the HBcAg polypeptide having the sequence shown in SEQ ID NO: 38). In further preferred embodiments, the cysteine residues at positions 48 and 107 of SEQ ID NO: 37 are mutated to serine. The invention further includes compositions comprising the corresponding polypeptides having amino acid sequences shown in WO 03/024481 (page 54 third paragraph to page 55 first paragraph), which also have above noted amino acid alterations. Further included within the scope of the invention are additional HBcAg variants which are capable of associating to form a capsid or VLP and have the above noted amino acid alterations. Thus, the invention further includes compositions and vaccine compositions, respectively, comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N-terminal leader sequence and modified with above noted alterations.

Compositions or vaccine compositions of the invention may comprise mixtures of different HBcAgs. Thus, these vaccine compositions may be composed of HBcAgs which differ in amino acid sequence. For example, vaccine compositions could be prepared comprising a “wild-type” HBcAg and a modified HBcAg in which one or more amino acid residues have been altered (e.g., deleted, inserted or substituted). Further, preferred vaccine compositions of the invention are those which present highly ordered and repetitive antigen array, wherein the antigen is a GnRH-peptide of the invention.

In a further preferred embodiment of the present invention, the at least one GnRH-peptide of the invention is bound to said virus-like particle by at least one covalent bond. Preferably, the at least one GnRH-peptide is bound to the virus-like particle by at least one covalent bond, said covalent bond being a non-peptide bond leading to a VLP-GnRH peptide array or conjugate, which is typically and preferably an ordered and repetitive array or conjugate. This GnRH-peptide-VLP array and conjugate, respectively, has typically and preferably a repetitive and ordered structure since the at least one, but usually more than one, GnRH-peptide of the invention is bound to the VLP and in an oriented manner. Preferably, more than 120, preferably more than 180, more preferably more than 270, and even more preferably more than 360 GnRH-peptides of the invention are bound to the VLP. The formation of a repetitive and ordered GnRH-VLP array and conjugate, respectively, is ensured by an oriented and directed as well as defined binding and attachment, respectively, of the at least one GnRH-peptide of the invention to the VLP as will become apparent in the following. Furthermore, the typical inherent highly repetitive and organized structure of the VLPs advantageously contributes to the ability to display the GnRH-peptide of the invention in a preferably highly ordered and repetitive fashion leading to a highly organized and repetitive GnRH-peptide-VLP array and conjugate, respectively. The GnRH-peptide is bound to the VLP via its N-terminus or C-Terminus, preferably via its N-terminus.

In a further preferred embodiment of the present invention, the virus-like particle comprises at least one first attachment site and wherein said at least one GnRH-peptide further comprises at least one second attachment site being selected from the group consisting of (i) an attachment site not naturally occurring with the at least one GnRH-peptide; and (ii) an attachment site naturally occurring with the at least one GnRH-peptide, and wherein said binding of the GnRH-peptide to the virus-like particle is effected through association between the first attachment site and the second attachment site, and wherein preferably the association is through at least one non-peptide bond.

The present invention discloses methods of binding of the at least one GnRH-peptide of the invention to VLPs. As indicated, in one preferred aspect of the invention, the GnRH-peptide of the invention is bound to the VLP by way of chemical cross-linking, typically and preferably by using a heterobifunctional cross-linker. Several hetero-bifunctional cross-linkers are known in the art. In preferred embodiments, the hetero-bifunctional cross-linker contains a functional group which can react with preferred first attachment sites, i.e. with the side-chain amino group of lysine residues of the VLP or at least one VLP subunit, respectively, and a further functional group which can react with a preferred second attachment site, i.e. a cysteine residue added to or engineered to be added to the GnRH-peptide of the invention, and optionally also made available for reaction by reduction. The first step of the procedure, typically called the derivatization, is the reaction of the VLP with the cross-linker. The product of this reaction is an activated VLP, also called activated carrier. In the second step, unreacted cross-linker is removed using usual methods such as gel filtration or dialysis. In the third step, the GnRH-peptide of the invention is reacted with the activated carrier, and this step is typically called the coupling step. Unreacted GnRH-peptide of the invention may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of an amide bond after reaction with the amino group and a thioether linkage with the cysteine. Another class of cross-linkers suitable in the practice of the invention is characterized by the introduction of a disulfide linkage between the GnRH-peptide of the invention and the VLP upon coupling. Preferred cross-linkers belonging to this class include for example SPDP and Sulfo-LC-SPDP (Pierce). The extent of derivatization of the VLP with cross-linker can be influenced by varying experimental conditions such as the concentration of each of the reaction partners, the excess of one reagent over the other, the pH, the temperature and the ionic strength. The degree of coupling, i.e. the amount of GnRH-peptides of the invention per subunits of the VLP, respectively, can be adjusted by varying the experimental conditions described above to match the requirements of the vaccine. Solubility of the GnRH-peptide of the invention may impose a limitation on the amount of GnRH-peptide of the invention that can be coupled on each subunit, and in those cases where the obtained vaccine would be insoluble reducing the amount of GnRH-peptide of the invention per subunit is beneficial.

A particularly favored method of binding of GnRH-peptide of the invention to the VLP is the linking of a lysine residue on the surface of the VLP, respectively, with a cysteine residue on the GnRH-peptide of the invention. Thus, in a preferred embodiment of the present invention, the first attachment site is a lysine residue and the second attachment site is a cysteine residue. In some embodiments, engineering of an amino acid linker containing a cysteine residue, as a second attachment site or as a part thereof, to the GnRH-peptide of the invention for coupling to the VLP, respectively, may be required. Alternatively, a cysteine may be introduced by addition to the GnRH-peptide of the invention. Alternatively, the cysteine residue may be introduced by chemical coupling.

In a further embodiment, the composition or vaccine composition, respectively, of the invention comprising a VLP and at least one GnRH peptide further comprises a linker, preferably an amino acid linker. Preferably, the linker comprises, or alternatively essentially consists of, or alternatively consists of the second attachment site. The selection of the amino acid linker will be dependent on the nature of the GnRH-peptide of the invention, on its biochemical properties, such as pI, charge distribution and glycosylation. Typically, flexible amino acid linkers are favored. Preferred embodiments of the amino acid linker are disclosed in WO 03/039225 on page 60, line 24 to page 61, line 11 (paragraphs 00179 and 00180), which are explicitly incorporated herein by way of reference. Preferred linkers of the invention are short linkers. Typically and preferably, the linkers of the invention do not essentially affect the immune response against GnRH. In a preferred embodiment, the linker of the invention comprises, essentially consists of, or consist of not more than 5 amino acids, preferably less than 5, even more preferably less than 4 and particularly preferred at most 3 amino acids. Further preferred linkers of the invention comprise or consist of less than 3 or less than 2 amino acids, preferably 1 amino acid. Preferably, the amino acid linker of the invention is selected from the group consisting of C, CG, CGG, GC, and GGC, preferably C or CGG.

The amino acid linker may be attached at the N-terminus or C-terminus of the GnRH peptide. In a preferred embodiment, the amino acid linker is attached at the N-terminus of the GnRH peptide or fragment or variant thereof, of the invention. Preferred amino acid linkers at the N-terminus of the GnRH peptide are CGG, CG, or C. Preferred amino acid linkers at the C-terminus of the GnRH peptide are GGCG (SEQ ID NO: 41), GGC, GGC-NH2 (“NH2” stands for amidation), GC, GC-NH2, C, or C-NH2 linkers. In general, glycine residues will be inserted between bulky amino acids and the cysteine to be used as second attachment site, to avoid potential steric hindrance of the bulkier amino acid in the coupling reaction. In a preferred embodiment, the GnRH peptide with the amino acid linker or second attachment site, respectively, has an amino acid sequence selected from the group consisting of SEQ ID NO:2, SEQ ID NO:3, SEQ ID NO:4, and SEQ ID NO:5.

Preferably, the at least one GnRH peptide of the invention comprises or consists of the amino acid sequence of SEQ ID NO: 1, and the linker or second attachment site, respectively, of the invention comprises or consists of C or CGG. In a preferred embodiment, the linker or second attachment site, respectively, is attached at the N-terminus of the at least one GnRH peptide and the GnRH peptide with the linker has an amino acid sequence selected from the group consisting of SEQ ID NO: 2 or SEQ ID NO: 4. Thus, in a particularly preferred embodiment of the invention, the composition or vaccine composition, respectively, comprises, or essentially consists of a GnRH peptide and linker of SEQ ID NO: 2 or 4 and an RNA phage, preferably bacteriophage Qβ.

The cysteine residue added to the GnRH-peptide of the invention has to be in its reduced state to react with the hetero-bifunctional cross-linker on the activated VLP, that is a free cysteine or a cysteine residue with a free sulfhydryl group has to be available. In the instance where the cysteine residue to function as binding site is in an oxidized form, for example if it is forming a disulfide bridge, reduction of this disulfide bridge with e.g. DTT, TCEP or β-mercaptoethanol is required.

Binding of the GnRH-peptide of the invention to the VLP by using a hetero-bifunctional cross-linker according to the preferred methods described above, allows coupling of the GnRH-peptide of the invention to the VLP in an oriented fashion. Other methods of binding the GnRH-peptide of the invention to the VLP include methods wherein the GnRH-peptide of the invention is cross-linked to the VLP using the carbodiimide EDC, and NHS. The GnRH-peptide of the invention may also be first thiolated through reaction, for example with SATA, SATP or iminothiolane. The GnRH-peptide of the invention, after deprotection if required, may then be coupled to the VLP as follows. After separation of the excess thiolation reagent, the GnRH-peptide of the invention is reacted with the VLP previously activated with a hetero-bifunctional cross-linker comprising a cysteine reactive moiety, and therefore displaying at least one or several functional groups, preferably one, reactive towards cysteine residues, to which the thiolated GnRH-peptide of the invention can react, such as described above. Optionally, low amounts of a reducing agent are included in the reaction mixture. In further methods, the GnRH-peptide of the invention is attached to the VLP using a homo-bifunctional cross-linker such as glutaraldehyde, DSG, BM[PEO]4, BS3, (Pierce Chemical Company, Rockford, Ill., USA) or other known homo-bifunctional cross-linkers with functional groups reactive towards amine groups or carboxyl groups of the VLP.

Other methods of binding the VLP to a GnRH-peptide of the invention include methods where the VLP is biotinylated, and the GnRH-peptide of the invention expressed as a streptavidin-fusion protein, or methods wherein both the GnRH-peptides of the invention and the VLP are biotinylated, for example as described in WO 00/23955. In this case, the GnRH-peptide of the invention may be first bound to streptavidin or avidin by adjusting the ratio of GnRH-peptide of the invention to streptavidin such that free binding sites are still available for binding of the VLP which is added in the next step. Alternatively, all components may be mixed in a “one pot” reaction. Other ligand-receptor pairs, where a soluble form of the receptor and of the ligand is available, and are capable of being cross-linked to the VLP or the GnRH-peptide of the invention, may be used as binding agents for binding the GnRH-peptide of the invention to the VLP. Alternatively, either the ligand or the receptor may be fused to the GnRH-peptide of the invention, and so mediate binding to the VLP chemically bound or fused either to the receptor, or the ligand respectively. Fusion may also be effected by insertion or substitution.

As already indicated, in a favoured embodiment of the present invention, the VLP is the VLP of a RNA phage, and in a more preferred embodiment, the VLP is the VLP of RNA phage Qβ coat protein.

One or several antigen molecules, i.e. GnRH-peptides of the invention, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit. This allows for the generation of a dense antigen array.

In a preferred embodiment of the invention, the binding and attachment, respectively, of the at least one GnRH-peptide of the invention to the virus-like particle is by way of interaction and association, respectively, between at least one first attachment site of the virus-like particle and at least one second attachment added to the GnRH-peptide of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the GnRH-peptide of the invention is bound via a cysteine residue, having been added to the GnRH-peptide of the invention, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost. In another preferred embodiment, VLPs are derived from fusion proteins of RNA phage coat proteins with a GnRH-peptide of the invention.

The use of the VLPs as carriers allows the formation of robust antigen arrays and conjugates, respectively, with variable antigen density. In particular, the use of VLPs of RNA phages, and hereby in particular the use of the VLP of RNA phage Qβ coat protein allows achievement of a very high epitope or antigen density. The preparation of compositions of VLPs of RNA phage coat proteins with a high epitope or antigen density can be effected by using the teaching of this application. In a preferred embodiment, the compositions and vaccines of the invention have an antigen density being from 0.05 to 4.0. The term “antigen density”, as used herein, refers to the average number of GnRH-peptide of the invention which is linked per subunit, preferably per coat protein, of the VLP, and hereby preferably of the VLP of a RNA phage. Thus, this value is calculated as an average over all the subunits or monomers of the VLP, preferably of the VLP of the RNA-phage, in the composition or vaccines of the invention. In a further preferred embodiment of the invention, the antigen density is, preferably between 0.1 and 4.0.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the preferred cross-linkers. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ VLPs. Thus, in another preferred embodiment of the present invention, the virus-like particle comprises, consists essentially of or alternatively consists of mutant Qβ coat proteins. Preferably these mutant coat proteins comprise or alternatively consist of an amino acid sequence selected from the group of a) Qβ-240 (Lys13-Arg; SEQ ID NO: 23) b) Qβ-243 (Asn 10-Lys; SEQ ID NO: 24), c) Qβ-250 (Lys2-Arg of SEQ ID NO: 25) d) Qβ-251 (Lys16-Arg of SEQ ID NO: 26); and e) Qβ-259″ (Lys2-Arg, Lys16-Arg of SEQ ID NO: 27). The construction, expression and purification of the above indicated Qβ coat proteins, mutant Qβ coat protein VLPs and capsids, respectively, are described in WO 02/056905. In particular is hereby referred to Example 18 of above mentioned application. In another preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing mutants and the corresponding A1 protein.

A particularly favored method of attachment of antigens to VLPs, and in particular to VLPs of RNA phage coat proteins is the linking of a lysine residue present on the surface of the VLP of RNA phage coat proteins with a cysteine residue naturally present or engineered on the antigen, i.e. the GnRH-peptide of the invention. In order for a cysteine residue to be effective as second attachment site, a sulfhydryl group must be available for coupling. Thus, a cysteine residue has to be in its reduced state, that is, a free cysteine or a cysteine residue with a free sulfhydryl group has to be available. In the instant where the cysteine residue to function as second attachment site is in an oxidized form, for example if it is forming a disulfide bridge, reduction of this disulfide bridge with e.g. DTT, TCEP or β-mercaptoethanol is required. The concentration of reductand, and the molar excess of reductant over antigen have to be adjusted for each antigen. A titration range, starting from concentrations as low as 10 μM or lower, up to 10 to 20 mM or higher reductant if required is tested, and coupling of the antigen to the carrier assessed. Although low concentrations of reductant are compatible with the coupling reaction as described in WO 02/056905, higher concentrations inhibit the coupling reaction, as a skilled artisan would know, in which case the reductant has to be removed by dialysis or gel filtration. Advantageously, the pH of the dialysis or equilibration buffer is lower than 7, preferably 6. The compatibility of the low pH buffer with antigen activity or stability has to be tested.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactants, and manipulation of the reaction conditions can be used to control the number of antigens coupled to VLPs of RNA phage coat proteins, and in particular to VLPs of Qβ coat protein.

Prior to the design of a non-natural second attachment site the position at which it should be fused, inserted or generally engineered has to be chosen. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

In preferred embodiments, the GnRH-peptide of the invention comprises an added single second attachment site or a single reactive attachment site capable of association with the first attachment sites on the VLPs or VLP subunits, respectively. This ensures a defined and uniform binding and association, respectively, of the at least one, but typically more than one, preferably more than 10, 20, 40, 80, 120, 150, 180, 210, 240, 270, 300, 360, 400, 450 GnRH-peptides of the invention to the VLP. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine- (as the first attachment site) and cysteine- (as a second attachment site) interaction, it is ensured, in accordance with this preferred embodiment of the invention, that only one added cysteine residue per GnRH-peptide of the invention is capable of binding and associating, respectively, with the VLP.

In some embodiments, engineering of a second attachment site onto the GnRH-peptide of the invention is achieved by the fusion of an amino acid linker containing an amino acid suitable as second attachment site according to the disclosures of this invention. Therefore, in a preferred embodiment of the present invention, an amino acid linker is bound to the GnRH-peptide, preferably, by way of at least one covalent bond. Preferably, the amino acid linker comprises, or alternatively consists of, the second attachment site. In a further preferred embodiment, the amino acid linker comprises a sulfhydryl group or a cysteine residue. In another preferred embodiment, the amino acid linker is cysteine. Some criteria of selection of the amino acid linker as well as further preferred embodiments of the amino acid linker according to the invention have already mentioned above.

In a further preferred embodiment of the invention, the at least one GnRH-peptide of the invention is fused to the virus-like particle. As outlined above, a VLP is typically composed of at least one subunit, typically and preferably said subunit is a coat protein or a mutant or a fragment thereof, assembling into a VLP. Thus, in again a further preferred embodiment of the invention, the GnRH-peptide of the invention is fused to at least one subunit of the virus-like particle or of a protein capable of being incorporated into a VLP generating a chimeric VLP-subunit GnRH-peptide protein fusion.

Fusion of GnRH-peptides of the invention can be effected by insertion into the VLP subunit sequence, or by fusion to either the N- or C-terminus of the VLP-subunit or protein capable of being incorporated into a VLP. Hereinafter, when referring to fusion proteins of a GnRH peptide to a VLP subunit, the fusion to either ends of the subunit sequence or internal insertion of the peptide within the subunit sequence are encompassed. Preferred embodiments are the fusion with the GnRH-peptide of the invention being at the N-terminus or C-terminus of the fusion polypeptide, i.e. fused via its C-terminus or N-terminus to the VLP subunit. Thus, the GnRH-peptide is fused via its N-terminus or C-terminus to the VLP, preferably via its N-terminus.

Fusion may also be effected by inserting sequences of the GnRH-peptide of the invention into a variant of a VLP subunit where part of the subunit sequence has been deleted, that are further referred to as truncation mutants. Truncation mutants may have N- or C-terminal, or internal deletions of part of the sequence of the VLP subunit. For example, the specific VLP HBcAg with, for example, deletion of amino acid residues 79 to 81 is a truncation mutant with an internal deletion. Fusion of GnRH-peptide of the invention to either the N- or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the sequence of the GnRH-peptide of the invention into the sequence of a VLP subunit, by substitution of part of the sequence of the VLP subunit with the sequence of the GnRH-peptide of the invention, or by a combination of deletion, substitution or insertions.

The chimeric GnRH-peptide-VLP subunit in general will be capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of and assembly to a VLP. In those embodiments, the proportion of chimeric VLP-subunits of total VLP subunits may be 1, 2, 5, 10, 20, 30, 40, 50, 60, 70, 80, 90, 95% or higher.

Flanking amino acid residues may be added to either end of the sequence of the GnRH-peptide of the invention, fulfilling the requirements as set out for fusion polypeptides of the invention above, to be fused to either end of the sequence of the subunit of a VLP, or for internal insertion of such peptidic sequence into the sequence of the subunit of a VLP. Glycine and serine residues are particularly favored amino acids to be used in the flanking sequences added to the GnRH-peptide of the invention to be fused. Glycine residues confer additional flexibility, which may diminish the potentially destabilizing effect of fusing a foreign sequence into the sequence of a VLP subunit.

In a specific embodiment of the invention, the VLP is a Hepatitis B core antigen VLP. Fusion proteins to either the N-terminus of HBcAg (Neyrinck, S. et al., Nature Med. 5:1157-1163 (1999)) or insertions in the so called major immunodominant region (MIR) have been described (Pumpens, P. and Grens, E., Intervirology 44:98-114 (2001)), WO 01/98333), and are preferred embodiments of the invention. Naturally occurring variants of HBcAg with deletions in the MIR have also been described (Pumpens, P. and Grens, E., Intervirology 44:98-114 (2001), which is expressly incorporated by reference in their entirety), and fusions to the N- or C-terminus, as well as insertions at the position of the MIR corresponding to the site of deletion as compared to a wt HBcAg are further embodiments of the invention. Fusions to the C-terminus have also been described (Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Habor Laboratory Press, Cold Spring Harbor, N.Y. (1989), Ho et al., Gene 77:51 (1989)).

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (PIR Database, Accession No. VCBPQB referring to Qβ CP and Accession No. AAA16663 referring to Qβ A1 protein) and bacteriophage fr (PIR Accession No. VCBPFR).

In a more preferred embodiment, the at least one GnRH-peptide of the invention is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins CP. The part of the A1 gene, 3′ of the UGA amber codon encodes the CP extension, which has a length of 195 amino acids. Insertion of the at least one GnRH-peptide of the invention between position 72 and 73 of the CP extension leads to further embodiments of the invention (Kozlovska, T. M., et al., Intervirology 39:9-15 (1996)). Fusion of a GnRH-peptide of the invention at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology, 39:9-15 (1996)), assembly of the particles displaying the fused epitopes typically requires the presence of both the A1 protein-GnRH-peptide fusion and the wt CP to form a mosaic particle. However, embodiments comprising virus-like particles, and hereby in particular the VLPs of the RNA phage Qβ coat protein, which are exclusively composed of VLP subunits having at least one GnRH-peptide of the invention fused thereto, are also within the scope of the present invention.

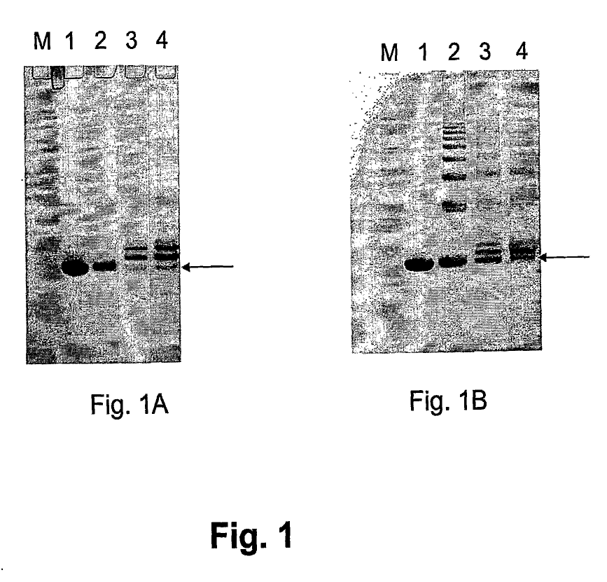

The production of mosaic particles may be effected in a number of ways. Kozlovska et al., Intervirolog, 39:9-15 (1996), describe two methods, which both can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:33-40 (1993))). In another approach, the CP gene stop codon is modified into UAA, and a second plasmid expressing the A1 protein-GnRH-peptide fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-GnRH-peptide fusion are encoded in a bicistronic manner, operatively linked to a promoter such as the Trp promoter, as described in FIG. 1 of Kozlovska et al., Intervirology, 39:9-15 (1996).

In a further embodiment, the GnRH-peptide of the invention is inserted between amino acid 2 and 3 (numbering of the cleaved CP, that is wherein the N-terminal methionine is cleaved) of the fr CP, thus leading to a GnRH-peptide-fr CP fusion protein. Vectors and expression systems for construction and expression of fr CP fusion proteins self-assembling to VLP and useful in the practice of the invention have been described (Pushko P. et al., Prot. Eng. 6:883-891 (1993)). In a specific embodiment, the sequence of the GnRH-peptide of the invention is inserted into a deletion variant of the fr CP after amino acid 2, wherein residues 3 and 4 of the fr CP have been deleted (Pushko P. et al., Prot. Eng. 6:883-891 (1993)).

Fusion of epitopes in the N-terminal protuberant β-hairpin of the coat protein of RNA phage MS-2 and subsequent presentation of the fused epitope on the self-assembled VLP of RNA phage MS-2 has also been described (WO 92/13081), and fusion of the GnRH-peptide of the invention by insertion or substitution into the coat protein of MS-2 RNA phage is also falling under the scope of the invention.

In another embodiment of the invention, the GnRH-peptides of the invention are fused to a capsid protein of papillomavirus. In a more specific embodiment, the GnRH-peptides of the invention are fused to the major capsid protein L1 of bovine papillomavirus type 1 (BPV-1). Vectors and expression systems for construction and expression of BPV-1 fusion proteins in a baculovirus/insect cells systems have been described (Chackerian, B. et al., Proc. Natl. Acad. Sci. USA 96:2373-2378 (1999), WO 00/23955). Substitution of amino acids 130-136 of BPV-1 L1 with a GnRH-peptide of the invention leads to a BPV-1 L1-GnRH-peptide fusion protein, which is a preferred embodiment of the invention. Cloning in a baculovirus vector and expression in baculovirus infected Sf9 cells has been described, and can be used in the practice of the invention (Chackerian, B. et al., Proc. Natl. Acad. Sci. USA 96:2373-2378 (1999), WO 00/23955). Purification of the assembled particles displaying the fused GnRH-peptides of the invention can be performed in a number of ways, such as for example gel filtration or sucrose gradient ultracentrifugation (Chackerian, B. et al., Proc. Natl. Acad. Sci. USA 96:2373-2378 (1999), WO 00/23955).

In a further embodiment of the invention, the GnRH-peptides of the invention are fused to a Ty protein capable of being incorporated into a Ty VLP. In a more specific embodiment, the GnRH-peptides of the invention are fused to the p1 or capsid protein encoded by the TYA gene (Roth, J. F., Yeast 16:785-795 (2000)). The yeast retrotransposons Ty1, 2, 3 and 4 have been isolated from Saccharomyces Cerevisiae, while the retrotransposon Tf1 has been isolated from Schizosaccharomyces Pombae (Boeke, J. D. and Sandmeyer, S. B., “Yeast Transposable elements,” in The molecular and Cellular Biology of the Yeast Saccharomyces: Genome dynamics, Protein Synthesis, and Energetics., p. 193, Cold Spring Harbor Laboratory Press (1991)). The retrotransposons Ty1 and 2 are related to the copia class of plant and animal elements, while Ty3 belongs to the gypsy family of retrotransposons, which is related to plants and animal retroviruses. In the Ty1 retrotransposon, the p1 protein, also referred to as Gag or capsid protein has a length of 440 amino acids. P1 is cleaved during maturation of the VLP at position 408, leading to the p2 protein, the essential component of the VLP.

Fusion proteins to p1 and vectors for the expression of said fusion proteins in Yeast have been described (Adams, S. E., et al., Nature 329:68-70 (1987)). So, for example, a GnRH-peptide of the invention may be fused to p1 by inserting a sequence coding for the GnRH-peptide of the invention into the BamH1 site of the pMA5620 plasmid (Adams, S. E., et al., Nature 329:68-70 (1987)). The cloning of sequences coding for foreign epitopes into the pMA5620 vector leads to expression of fusion proteins comprising amino acids 1-381 of p1 of Ty1-15, fused C-terminally to the N-terminus of the foreign epitope. Likewise, N-terminal fusion of GnRH-peptides of the invention, or internal insertion into the p1 sequence, or substitution of part of the p1 sequence is also meant to fall within the scope of the invention. In particular, insertion of GnRH-peptides of the invention into the Ty sequence between amino acids 30-31, 67-68, 113-114 and 132-133 of the Ty protein p1 (EP0677111) leads to preferred embodiments of the invention.

Further VLPs suitable for fusion of GnRH-peptides of the invention are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0020416B1).

In one embodiment of the invention, the VLP is a VLP of an AP205-bacteriophage and the linkage of said VLP and said at least one GnRH peptide is by way of fusion between said GnRH peptide and the AP205 coat protein or a mutant or fragment thereof. Said fusion can be either direct or indirect via a spacer or linker. The GnRH peptide can be either fused to the N- or to the C-terminus, preferably to the C-terminus of the AP205 coat protein or a mutant or fragment thereof. The term “mutant coat protein of a virus”, and hereby preferably the term “AP205 mutant coat protein”, as used herein, should refer to a polypeptide having an amino acid sequence which (i) differs by at least one amino acid with respect to the amino acid sequence of the coat protein or coat proteins of the virus, and hereby preferably to the coat protein of AP205-bacteriophage having the SEQ ID NO: 39; and (ii) has an identity to the coat protein or coat proteins, and hereby preferably to SEQ ID NO: 39, of at least 85%, preferably 90%, more preferably 92%, even more preferably 95%, still more preferably 97%; and, preferably (iii) is capable of assembling into a virus-like particle. The term “fragment of a coat protein of a virus”, and hereby preferably the term “fragment of AP205 coat protein”, as used herein, should refer to a polypeptide having an amino acid sequence which (i) has a length of at least 70%, preferably at least 80%, more preferably at least 90%, even more preferably at least 95% of the length of the coat protein or coat proteins of the virus, and hereby preferably of the length of SEQ ID NO: 39; and, preferably (ii) is capable of assembling into a virus-like particle; and, preferably (iii) has an identity to the coat protein or coat proteins, and hereby preferably to SEQ ID NO: 39, of at least 50%, preferably 60%, preferably 70%, more preferably 80%, even more preferably 90%, still more preferably 95%. Preferably, the fragment is obtained by at least one, preferably one, internal deletion, at least one, preferably one or two, even more preferably one, truncation or at least one, preferably one or two, even more preferably one combination thereof.

Examples of chimeric VLPs suitable for the practice of the invention are also those described in Intervirology 39:1 (1996). Further examples of VLPs contemplated for use in the invention are: HPV-1, HPV-6, HPV-11, HPV-16, HPV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and Norwalk virus have also been made, and chimeric VLPs of those VLPs are also within the scope of the present invention.

GnRH-peptides of the invention can be produced by expression of DNA encoding GnRH-peptide of the invention under the control of a strong promotor. Various examples hereto have been described in the literature and can be used, possibly after modifications, to express GnRH-peptide of the invention of any desired species, preferably in the context of fusion polypeptides, e.g. a fusion with GST or DHFR.

Such GnRH-peptides of the invention can be produced using standard molecular biological technologies where the nucleotide sequence coding for the fragment of interest is amplified by PCR and is cloned as a fusion to a polypeptide tag, such as the histidine tag, the Flag tag, myc tag or the constant region of an antibody (Fc region). By introducing an enterokinase cleavage site between the GnRH-peptide of the invention and the tag, the GnRH-peptide of the invention can be separated from the tag after purification by digestion with enterokinase. In another approach the GnRH-peptide of the invention can be synthesized in vitro with or without a phosphorylation-modification using standard peptide synthesis reactions known to a person skilled in the art.

Guidance on how to modify GnRH-peptide of the invention, in particular, for binding to the virus-like particle is given throughout the application. Immunization against GnRH using the inventive compositions comprising a GnRH-peptide of the invention bound to a VLP may provide a way of treating GnRH-related disorders.

In a still further preferred embodiment of the present invention, the GnRH-peptide of the invention further comprises at least one second attachment site not naturally occurring within said GnRH-peptide of the invention. In a preferred embodiment, said attachment site comprises an amino acid linker of the invention, preferably a linker sequence of C, CG, GC, GGC or CGG.

Thus, the present invention surprisingly shows that GnRH peptides coupled to VLPs, preferably through short linkers which do not affect the immune response against GnRH are able to induce strong immune responses, in particular strong antibody responses, leading to high antibody titer against the self antigen GnRH, and thus resulting in reduced testosterone levels, testicular atrophy and infertility. Therefore, vaccines of the invention are not linker dependent. This is in particular advantageous over the solution of the prior art, since the composition or vaccine, respectively, of the invention does not need a specific linker to positively affect the immune response and does not need to analyze or identify the linker for every different peptide. In addition, such short linker sequences may not divert the antibody responses away from the GnRH sequence, resulting in increased antibody responses to GnRH compared to prior art linkers due to the elimination of a competing linker-specific antibody response. In addition, using short linker sequences of the invention, such as C, CG, GC, GGC or CGG, preferably C, overcomes the problem of inducing T cell response. The probability to induce a T cell response dramatically increases with increased linker length. Thus, vaccination against self-antigens by using compositions or vaccines, respectively, of the invention, eliminates the problem of undesired inflammatory and/or cytotoxic immune responses.

Some of the very preferred GnRH-peptides of the invention are described in the Examples. These peptides comprise an N- or C-terminal cysteine residue as a second attachment added for coupling to VLPs. These very preferred short GnRH-peptides of the invention are capable of having a much enhanced immunogenicity when coupled to VLP.

In further preferred embodiments of the invention, the GnRH-peptide consists of a peptide with a length of 5 to 10 amino acid residues, and are, furthermore, capable of overcoming possible safety issues that arise when targeting self-proteins, as shorter fragment are much more less likely to contain T cell epitopes. Typically, the shorter the peptides, the safer with respect to T cell activation.

The invention further relates to the use of the modified VLP of the invention or of a composition of the invention or of the pharmaceutical composition of the invention for the preparation of a medicament for the treatment of GnRH-related diseases. The treatment is preferably a therapeutic treatment or alternatively a prophylactic treatment. Preferred GnRH-related diseases or conditions that are treated are any diseases or conditions in human or other mammals which are brought on or aggravated by GnRH, such as for example fertility, gonadal steroid hormone dependent cancer, breast cancer, uterine and other gynecological cancers, endometriosis, uterine fibroids, prostate cancer, benign prostatic hypertrophy, boar taint in pork, beef or sheep, meat quality of male animals kept for meat production, gonadal steroid hormone related behaviour in animals, for example aggression or sexual activity, and reproduction in wild life animals, modulation of thymus function and T-lymphocyte production in lymphocyte depleted individuals. In a preferred embodiment, the condition treated is the meat quality of male animals kept for meat production, preferably in rams, boars or bulls, very preferably in boars.

In a preferred embodiment the GnRH-peptide of the modified VLP to be used is derived from mammalian GnRH. Such conjugates are preferably to be used for the manufacture of a medicament for the treatment of GnRH-related diseases or conditions, preferably of fertility, gonadal steroid hormone dependent cancer, prostate cancer, boar taint in pork, beef or sheep, meat quality of male animals kept for meat production, gonadal steroid hormone related behaviour in animals, for example aggression or sexual activity, and reproduction in wild life animals, modulation of thymus function and T-lymphocyte production in lymphocyte depleted individuals. In a preferred embodiment, the condition treated is the meat quality of male animals kept for meat production, preferably in rams, boars or bulls, very preferably in boars.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

### Example 1

### Coupling of GnRH Peptides to Qβ VLPs

The following peptides comprising amino acid 1-10 of GnRH (pEHWSYGLRPG-NH2: SEQ ID NO:1), extended with either a cysteine as attachment site for coupling or with two glycine residues plus a cysteine residue as attachment site, were chemically synthesized:

| | | |
|---|---|---|
| CGG-GnRH | CGGEHWSYGLRPG-NH2 | (SEQ ID NO: 2) |
| GnRH-GGC | pEHWSYGLRPGGGC | (SEQ ID NO: 3) |
| C-GnRH | CEHWSYGLRPG-NH2 | (SEQ ID NO: 4) |
| GnRH-C | pEHWSYGLRPGC | (SEQ ID NO: 5) |

Peptides were coupled to Qβ VLPs as described below.

Qβ VLPs (1 mg/ml) in 20 mM Hepes pH7.2 were derivatized with an 18 fold molar excess of SMPH for 0.5 h at 25° C. Reactions were subsequently dialysed against 20 mM Hepes pH7.2 and coupled with a 10 fold molar excess of either CGG-GnRH (SEQ ID NO:2) or GnRH-GGC (SEQ ID NO:3) peptide (10 mM in DMSO) by incubation on a thermoshaker for 2 h at 25° C. Reactions were dialysed overnight against 20 mM Hepes pH7.2 to remove uncoupled peptide.

Coupling of peptides C-GnRH (SEQ ID NO:4) and GnRH-C (SEQ ID NO:5) was performed by derivatizing Qβ VLPs (2.8 mg/ml) in 20 mM Hepes pH7.2 with a 20 fold molar excess SMPH (50 mM in DMSO) for 0.5 h at 25° C. followed by overnight dialysis against 20 mM Hepes pH7.2. Subsequently, the derivatized Qβ was incubated on a thermoshaker for 2 h at 25° C. with either 2.5 fold or 7 fold molar excess of peptide (5 mM in DMSO), respectively. Reactions were dialysed against 20 mM Hepes pH7.2 overnight to remove uncoupled peptide.

The Qβ-GnRH coupling products were centrifuged and supernatants were analysed on SDS-PAGE gel under reducing conditions. Qβ-GnRH coupling products were named Qβ-CGG-GnRH, GnRH-GGC-Qβ, Qβ-C-GnRH and GnRH-C-Qβ according to the respective peptides (SEQ ID NO: 2, 3, 4 and 5) that were used for coupling.

FIG. 1 shows successful coupling of GnRH peptides (SEQ ID NO:2, 3, 4 and 5) to Qβ. Multiple coupling bands above the uncoupled monomer (arrow) consist of one, two, three and four peptides coupled to the Qβ monomer.

### Example 2

### Neutralising Antibody Response of Mice Immunized with Qβ-GnRH VLPs

Immunization of Mice with Qβ-GnRH VLPs for Suppression of Testicular Function

Recombinantly produced Qβ VLPs were used for immunization after coupling to GnRH peptides as described above. Eight week old male C57BV/6 mice (five mice per group) were immunized with 50 μg of Qβ-CGG-GnRH on day 0 and day 28, either with or without alum as adjuvant. Qβ-CGG-GnRH vaccine with high coupling efficiency was used. Control mice received Qβ. Anti-GnRH antibody titers and testosterone levels were measured in these mice. On day 70 after immunization, mice were killed and testes weight was determined.

### Anti-GnRH Antibody Titers in Mice

Serum was collected from immunized mice and control mice at various time points during the experiment. Anti-GnRH IgG antibody titer was determined by ELISA as follows. ELISA plates (Nunc Maxisorp) were coated with 10 μg/ml of CGG-GnRH (SEQ ID NO:2) coupled to RNase. Plates were blocked with 2% BSA and incubated with serial dilutions of mice sera. As a control, pre-immune sera was also tested. As a secondary antibody, 1:1000 dilution of goat anti-mouse IgG (H+L)−HRPO (Jackson ImmunoResearch Cat no 115-035-146) was used. After substrate addition and stopping the color reaction, optical density (OD) at 450 nm was determined on an ELISA reader (BioRad Benchmark). Using these data the serum dilution resulting in half the maximum OD450 was calculated

Table 1 shows that in male mice immunized with Qβ-CGG-GnRH, an average titer of 8513 was reached on day 21 and that upon boosting an average titer of 12716 was reached on day 47. In addition, when using alum as an adjuvant, average titers were even reaching 100.000 at days 47 and 54. These results clearly show that Qβ-CGG-GnRH is able to induce a high antibody titer against GnRH.

TABLE 1
Average 50% ODmax titer of five male mice immunized with 50 μg
Qβ-CGG-GnRH on day 0 and day 28

| | | Qβ-CGG-GnRH + |
|---|---|---|
| days post | Qβ-CGG-GnRH | Alum |
| immunization | 50% ODmax titer | 50% ODmax titer |
| day 21 | 8513 | 36152 |
| day 28 | 3409 | 41539 |
| day 47 | 12716 | 102369 |
| day 54 | 11259 | 111599 |
| day 70 | 5201 | 56152 |

### Testosterone Levels in Mice Serum

Serum was collected from immunized mice and control mice at various time points during the above described experiment. Using a Testosterone-Elisa (IBL, Hamburg, Germany) the testosterone levels in individual mice sera were determined.

Table 2 shows that in mice immunized with Qβ-CGG-GnRH, the average testosterone level is greatly suppressed (<0.5 ng/ml) on day 47 after immunization, with levels being lower than 0.2 ng/ml on day 70. In mice immunized with Qβ-CGG-GnRH complemented with alum, average testosterone level has dropped to <0.2 ng/ml on day 47. Control mice showed strong natural variation in testosterone levels with the average levels being approximately 10 fold higher than the levels in Qβ-CGG-GnRH immunized mice. This clearly demonstrates neutralising activity of the induced antibody response.

TABLE 2
Average testosterone levels (ng/ml) in male mice immunized
with Qβ-CGG-GnRH alone and Qβ-CGG-GnRH + alum

| | | Qβ-CGG- | |
|---|---|---|---|
| | Qβ-CGG-GnRH | GnRH + Alum | Qβ + Alum |
| | average ± sd | average ± sd | average ± sd |
| days post | testosterone | testosterone | testosterone |
| immunization | ng/ml | ng/ml | ng/ml |
| day 0 | 5.42 ± 4.80 | 3.83 ± 2.78 | 0.83 ± 0.24 |
| day 14 | 5.01 ± 2.54 | 3.15 ± 5.03 | 6.79 ± 7.47 |
| day 21 | 1.36 ± 1.65 | 0.60 ± 0.38 | 2.18 ± 2.86 |
| day 28 | 1.42 ± 0.84 | 0.50 ± 0.65 | 1.85 ± 3.42 |
| day 47 | 0.44 ± 0.89 | 0.14 ± 0.16 | 3.10 ± 4.27 |
| day 54 | 0.34 ± 0.58 | 0.14 ± 0.05 | 0.89 ± 0.69 |
| day 70 | 0.17 ± 0.21 | 0.17 ± 0.18 | 4.27 ± 6.77 |

### Testes Weight

Mice were sacrificed on day 70 and testes were removed and weighed, before fixing in 4% formaldehyde.

Table 3 shows the strongly reduced testes weight of Qβ-CGG-GnRH immunized mice on day 70. On average a greater than 50% reduction in testes weight was obtained in comparison to controls, while mice receiving Qβ-CGG-GnRH with alum showed a testes weight reduction of 75%. This clearly shows neutralising activity of the induced antibody response.

TABLE 3
Average testes weights of male mice immunized with
Qβ-CGG-GnRH with or without Alum, sacrificed on day 70

| | Average | |
|---|---|---|
| | testes weight | standard deviation of |
| Group | on day 70 (gram) | testes weight on day 70 |
| Qβ-CGG-GnRH | 0.091 | 0.054 |
| Qβ-CGG-GnRH + alum | 0.052 | 0.014 |
| Qβ + alum | 0.199 | 0.012 |

### Example 3

### Reduced Fertility of Mice Immunized with Qβ-GnRH VLPs

Immunization of Mice with Qβ-GnRH VLPs for Suppression of Fertility

Recombinantly produced Qβ VLPs were used for immunization after coupling to GnRH peptides as described above. Male and female C57B1/6 mice (8 weeks old) were immunized with 50%1 g of Qβ-CGG-GnRH on day 0, day 28 and day 42. On day 54 after immunization, mice were mated with untreated mice of the same age and control matings were performed with mice having received Qβ VLP only. After a period of 35 days, mice were separated and mating was repeated on day 120 after initial immunization. Litter size, antibody titer, testosterone levels were determined.

Table 4 shows that Qβ-CGG-GnRH immunized female mice were unable to produce any offspring (0 out of 10 matings), while control mice showed offspring production in 10 out of 10 matings. Also in the second mating round, no offspring was produced. The Qβ-CGG-GnRH immunized male mice showed a reduced percentage of succesfull matings (3 out of 10) while in the second mating the proportion was higher (5 out of 9) which shows reversibility of the neutralising effect of the induced GnRH antibody response.

TABLE 4
Number of successful matings (producing progeny) per total of
initialized matings.

Successful matings

| Immunization | 1st mating | 2nd mating |
|---|---|---|
| Qβ-CGG-GnRH female | 0/10 | 0/10 |
| Qβ-CGG-GnRH male | 3/10 | 5/9 |
| Qβ controls | 10/10 | 7/10 |

### Example 4

### Antibody Responses of Mice Immunized with Qb-CGG-GnRH are Higher than Antibody Responses Elicited Against GnRH-GGC-Qb

Serum was collected from male C57B1/6 mice on week 3 after subcutaneous immunization with 50 μg of either Qb-CGG-GnRH or GnRH-GGC-Qb. Testing the sera for GnRH antibody titers shows that N-terminal coupled peptide (Table 5, Qb-CGG-GnRH) produced higher antibody titers than C-terminal coupled peptide (Table 5, GnRH-GGC-Qb).

TABLE 5
ELISA titers (50% ODmax) of male mice immunized with 50 μg
vaccine. Titers are tested against CGG-GnRH and GnRH-GGC
coupled to RNase.

| vaccine | ELISA coating | week 3 titer |
|---|---|---|
| Qb-CGG-GnRH | CGG-GnRH | 11269 |
| GnRH-GGC-Qb | GnRH-GGC | 5975 |

For testing the efficient antibody response elicited by a short N-terminal cysteine linker, mice are immunized with 50 μg Qb-C-GnRH. Serum is taken at 3 weeks after immunization and is boosted with 50 μg Qb-C-GnRH and antibody responses are determined by ELISA. At 70 days post immunization, mice are sacrificed and testosterone levels and testes weights are determined.

### Example 5

### Coupling GnRH Peptides to VLPs Using Different Linker Sequences

Peptides (SEQ ID NO: 6-9 and 29-33, see Example 7), comprising an added N- or C-terminal cysteine residue with either one, two, three or no glycine residues present between peptide and cysteine, typically and preferably peptides of SEQ ID NO: 2 or 4, are coupled to VLPs, typically and preferably to Qβ in the following.

Recombinantly produced Qβ VLPs (2 mg/ml) are derivatized in 50 mM NaCl, 20 mM Hepes pH7.2 with a 20 fold molar excess of SMPH (Pierce) for 0.5 h at 25° C., followed by 2×2 h dialysis against 20 mM Hepes pH7.2 at 4° C., using 10.000 MWCO dialysis tubing, to remove unreacted SMPH. GnRH peptide (SEQ ID NO: 6-9 and 29-33) is added in a 7 fold molar excess and allowed to react for 2 h in a thermomixer at 25° C. Reactions are dialysed overnight against 20 mM Hepes pH7.2 to remove uncoupled peptide. Qβ-GnRH coupling products are centrifuged and supernatants are analysed on SDS-PAGE gel under reducing conditions. Gels are stained with Coomassie Blue.

### Example 6

### Immunization with GnRH Peptides Coupled to VLPs Using Different Linker Sequences

Male and female C57B1/6 mice (8 weeks of age) are immunized with GnRH peptides coupled to VLPs as described in example 5. 50-100 μg of VLP-GnRH either with alum, or emulsified in IFA or with no adjuvant, is injected subcutaneously at day 0. Mice are subsequently boosted as required. Blood is collected from mice at various time points during the experiment. For testing the efficacy of the VLP-GnRH immunization on inhibiting fertility, immunized mice are mated with not immunized mice and the percentage of successful matings in a group is determined. Anti-GnRH IgG antibody titers and testosterone levels are measured in sera from these mice. At the termination of the experiment, male mice are killed and testes weights are determined.

Throughout the example section, the term VLP-GnRH shall refer to a composition comprising a VLP, preferably RNA-phage, more preferably Qβ, and at least one GnRH peptide, preferably a GnRH peptide of any one of SEQ ID NO: 1-9 or 29-36, more preferably a GnRH Peptide of SEQ ID NO: 2 or 4.

### Example 7

### Coupling of GnRH Peptides Comprising the N-Terminal, resp. the C-Terminal Parts of the GnRH Sequence

The following GnRH peptides comprising part of the GnRH peptide (SEQ ID NO:1) are chemically synthesized:

| | | |
|---|---|---|
| GnRH2-10 | HWSYGLRPG | (SEQ ID NO: 6) |
| GnRH3-10 | WSYGLRPG | (SEQ ID NO: 7) |
| GnRH4-10 | SYGLRPG | (SEQ ID NO: 8) |
| GnRH5-10 | YGLRPG | (SEQ ID NO: 9) |
| GnRH1-9 | EHWSYGLRP | (SEQ ID NO: 29) |
| GnRH1-8 | EHWSYGLR | (SEQ ID NO: 30) |
| GnRH1-7 | EHWSYGL | (SEQ ID NO: 31) |
| GnRH1-6 | EHWSYG | (SEQ ID NO: 32) |
| GnRH1-5 | EHWSY | (SEQ ID NO: 33) |

Peptides (SEQ ID NO: 6-9 and 29-33), comprising a N- or C-terminal linker sequence as specified in Example 5 are coupled to VLPs as described for Qβ in the following.

Recombinantly produced Qβ VLPs (2 mg/ml) are derivatized in 50 mM NaCl, 20 mM Hepes pH7.2 with a 20 fold molar excess of SMPH (Pierce) for 0.5 h at 25° C., followed by 2×2 h dialysis against 20 mM Hepes pH7.2 at 4° C., using 10.000 MWCO dialysis tubing, to remove unreacted SMPH. GnRH peptide is added in a 7 fold molar excess and allowed to react for 2 h in a thermomixer at 25° C. Reactions are dialysed overnight against 20 mM Hepes pH7.2 to remove uncoupled peptide. Qβ-GnRH coupling products are centrifuged and supernatants are analysed on SDS-PAGE gel under reducing conditions. Gels are stained with Coomassie Blue.

### Example 8

### Immunization of Mice Immunized with GnRH Fragments Coupled to VLP

Male and female C57B1/6 mice (8 weeks of age) are immunized with VLP-GnRH conjugates described in example 7, with alum, emulsified in IFA or with no adjuvant. 50-100 μg of VLP-GnRH is injected subcutaneously at day 0. Mice are subsequently boosted as required. Blood is collected from mice at various time points during the experiment. For testing the efficacy of the VLP-GnRH immunization on inhibiting fertility, immunized mice are mated with not immunized mice and the percentage of successful matings in a group is determined. Anti-GnRH IgG antibody titers and testosterone levels are measured in sera from these mice. At the termination of the experiment, male mice are killed and testes weights are determined.

### Example 9

### Immunization of Mammals with Qβ-GnRH Against Gonadal Steroid Hormone Dependent Cancers

### Breast Cancer

The effects of the Qβ-GnRH vaccine are studied on the growth of estrogen-dependent breast tumors, tested with a subline of the MCF-7 human breast cancer cell line generated (Mcf7B (BIM)) tumors in nude mice. The method of passive immunization is used in the experiments on immuno-incompetent nude mice, using anti-GnRH antibodies produced in rabbits or mice or rats immunized with Qβ-GnRH vaccine (comprising any peptide of SEQ ID NOs: 2-9 and 29-33) as immunogen. The method comprises the administration of anti-GnRH antibodies to mice bearing detectable tumors. Various positive controls are used in the following experiments including, the widely used and accepted therapy for breast cancer using the anti-estrogen Tamoxifen; a GnRH analog superagonist peptide, decapeptide, also known as decapeptyl which inhibits the release of LH and FSH from the gonadotrophs. The effects of placebo, phosphate-buffered saline solution (PBS), estradiol (E2) and anti-VLP antibodies in the human breast tumor xenografts are also tested.

In the experiments, breast tumors are grown in donor nude mice from Mcf7 (B1M) breast cancer cell line. After 7-8 weeks, the tumors are grafted to 62 female nude mice. Following 3 to 4 weeks, the tumor xenografts are evaluated to determine if the size of the tumor is large enough to initiate the therapy. On day 30 of the experiments, two mice are bled by heart puncture for serum antibody studies and tumors are harvested, measured and frozen for receptor studies, etc. The remaining mice are randomly separated into six groups of ten mice by tumor size.

Group 1 receives 0.5 ml of phosphate buffered saline solution administered i.p. twice weekly; Group 2 receives anti-VLP purified antibodies, 0.25 mg/0.5 ml, i.p. twice weekly; Group 3 receives anti-GnRH purified antibodies 0.35 mg/0.5 ml i.p. twice weekly; Group 4 receives 5 mg of Tamoxifen in a pellet implanted subcutaneously which is sufficient for 60 days; Group 5 mice receives a placebo pellet for 60 days; and Group 6 mice receives 0.72 mg of estradiol (E2) pellet implanted subcutaneously which is sufficient for 60 days.

Mice are evaluated on a twice per week basis regarding tumor progression, and removed from the studies when the tumors reach a size of 200 mm². Efficacy of the immunization is measured by ability to inhibit tumor growth.

### Prostate Cancer

The effects of the Qβ-GnRH vaccine are studied on the growth of the CWR22 androgen-dependent human prostate tumor cell line generated tumors in nude mice. The method of passive immunization is used in the experiments on immuno-incompetent nude mice, using anti-GnRH antibodies produced in rabbits, mice or rats immunized with Qβ-GnRH vaccine comprising the GnRH peptide of any of SEQ ID NOs: 2-9 and/or 29-33 as immunogen. The method comprises the administration of anti-GnRH antibodies to mice bearing detectable tumors. Various positive controls are used in the following experiments including Tamoxifen; a GnRH analog superagonist peptide, decapeptide, also known as decapeptyl which inhibits the release of LH and FSH from the gonadotrophs. The effects of placebo, phosphate-buffered saline solution (PBS), testosterone and anti-VLP antibodies in the human breast tumor xenografts are also tested.

In the experiments, tumors are grown in donor nude mice from CWR22 prostate cancer cell line. After 7-8 weeks, the tumors are grafted to 62 male nude mice. Following 3 to 4 weeks, the tumor xenografts are evaluated to determine if the size of the tumor is large enough to initiate the therapy. On day 21 after initiation of the therapy, the approximately time point at which the control animals have large tumors requiring sacrificing the animals, all of the mice are killed 6 h after the final injection and the tumors and spleens were removed. Mice are bled by heart puncture for serum antibody studies. Tumors are measured and are flash-frozen in liquid nitrogen or fixed in formalin and embedded in paraffin.

Group 1 receives 0.5 ml of phosphate buffered saline solution administered i.p. twice weekly; Group 2 receives anti-VLP purified antibodies, 0.25 mg/0.5 ml, i.p. twice weekly; Group 3 receives anti-GnRH purified antibodies 0.35 mg/0.5 ml i.p. twice weekly; Group 4 receives 5 mg of Tamoxifen in a pellet implanted subcutaneously which is sufficient for 21 days; Group 5 mice receives a placebo pellet for 60 days; and Group 6 mice receives 0.72 mg of testosterone pellet implanted subcutaneously which is sufficient for 21 days.

Mice are evaluated on a twice per week basis regarding tumor progression. Efficacy of the immunization is measured by ability to inhibit tumor growth.

### Example 10

### Immunization of Male Pigs with Qβ-GnRH for the Prevention of Boar Taint in Meat

The Qβ-GnRH vaccine (comprising any peptide of SEQ ID NOs: 2-9 and 29-33) is given to male pigs to prevent the occurrence of boar taint in the meat while circumventing the need for surgical castration. The amount of Qβ-GnRH vaccine is preferably in the range of 50-1000 μg and given either subcutaneously, or intramuscular or through other routes of immunization with or without the use of an adjuvant at 8 weeks before slaughter. A booster immunization is given at 4 weeks before slaughter. Alternatively one immunization only is given at 4-8 weeks before slaughter. Boar taint compounds skatole and androstenone are measured in fat samples taken at slaughter, testes weight is determined and anti-GnRH IgG titers are measured from serum.

### Example 11

### Immunization of Mammals with Qβ-GnRH as a Means of Immunocastration and Immunosterilisation for Fertility Management and for Controlling Gonadal Steroid Hormone Related Behaviour

Female cats are immunized with Qβ-GnRH (comprising any peptide of SEQ ID NOs: 2-9 and 29-33) as an alternative to surgical sterilization. The amount of Qβ-GnRH vaccine is preferably in the range of 25-500 μg and given subcutaneously with or without the use of an adjuvant at 6-12 months of age. A booster immunization is given 28 days post immunization. Alternatively one immunization only is given. To determine efficacy, anti-GnRH IgG titers are measured from serum and female cats are mated to male cats.

Male cats are immunized with Qβ-GnRH (comprising any peptide of SEQ ID NOs: 2-9 and 29-33) as an alternative to surgical castration for preventing unwanted progeny and/or gonadal steroid hormone related behaviour. The amount of Qβ-GnRH vaccine is preferably in the range of 25-500 μg and given subcutaneously with or without the use of an adjuvant at 6-12 months of age. A booster immunization is given 28 days post immunization. Alternatively one immunization only is given. To determine efficacy, testes sizes were measured at 16 weeks post boost in male controls and male vaccinates, by reference to orchidometer beads. Anti-GnRH IgG titers and testosterone levels are measured from serum and male cats are mated to female cats. Analysis of these data can demonstrate which formulation is able to prevent the development and maintenance of reproductive organs.

As a means of immunocastration and immunosterilisation Beagle/Foxhound cross male dogs and bitches are immunized with a formulation comprising Qβ-GnRH (comprising any peptide of SEQ ID NOs: 2-9 and 29-33) either with adjuvant or without adjuvant. Dogs are 6-10 months of age at the time of initial immunization. Control dogs are not immunized. Immunizations are at 0 days with a boost immunization at 28 days post immunization. The dose of vaccine is preferably in the range of 50-500 μg Qβ-GnRH. Blood samples are taken at monthly intervals after the boost immunization, and antibody titers measured by ELISA. Titers are measured by ELISA.

At 16 weeks post boost, testes sizes were measured in male controls and male vaccinates, by reference to orchidometer beads. Analysis of these data can demonstrate which formulation is able to prevent the development and maintenance of reproductive organs. To determine efficacy female dogs are mated to male dogs. Of significance in the analysis is the demonstration that the preferred formulation is able to give duration of the antibody response and high responder rate.

### Example 12

Immunization of mammals with Qβ-GnRH as a means of immunocastration and immunosterilisation for fertility management, improvement of meat quality and reduction of gonadal steroid hormone related behaviour

### Immunosterilisation and Immunocastration in Sheep

Female sheep are immunized with Qβ-GnRH (comprising any peptide of SEQ ID NOs: 2-9 and 29-33) as a means of immunosterilization. The amount of Qβ-GnRH vaccine is preferably in the range of 25-1000 μg and given subcutaneously with or without the use of an adjuvant at 6-12 months of age A booster immunization is given 28 days post immunization. Alternatively one immunization only is given. Anti-GnRH IgG titers are measured from serum and blood progesterone concentrations are measured weekly from 3 weeks before immunization until 30 weeks after. Comparing of progesterone concentrations in immunized and control sheep can demonstrate a cessation of the oestrus cycle.

The Qβ-GnRH vaccine (comprising any peptide of SEQ ID NOs: 2-9 and 29-33) is given to rams as an alternative for surgical castration. The amount of Qβ-GnRH vaccine is preferably in the range of 50-1000 μg and given either subcutaneously, or intramuscular or through other routes of immunization with or without the use of an adjuvant at three months of age. A booster immunization is given at 4 weeks post immunization. Alternatively one immunization only is given at three months. Anti-GnRH IgG titers are measured every two weeks from serum, and testes size is monitored in controls and vaccinates, by reference to orchidometer beads.

### Immunocastration in Bulls

The Qβ-GnRH vaccine (comprising any peptide of SEQ ID NOs: 2-9 and 29-33) is given to bulls used for meat production as an alternative for surgical castration. The amount of Qβ-GnRH vaccine is preferably in the range of 50-500 μg and given either subcutaneously, or intramuscular or through other routes of immunization with or without the use of an adjuvant at 8 months of age. A booster immunization is given at 4 weeks post immunization. Alternatively one immunization only is given at 8 months. Testes size is monitored and anti-GnRH IgG titers are measured from serum.

### Immunosterilization and Castration in Horses

Stallions and mares are immunized with Qβ-GnRH (comprising any peptide of SEQ ID NOs: 2-9 and 29-33) as a means of immuno-castration and immuno-sterilization and for preventing unwanted gonadal steroid hormone related behaviour. The amount of Qβ-GnRH vaccine is preferably in the range of 25-500 μg and given subcutaneously with or without the use of an adjuvant at 2 years of age. A booster immunization is given 28 days post immunization. Alternatively one immunization only is given. To determine efficacy, anti-GnRH IgG titers testosterone and progesterone levels are measured from serum gonadal steroid hormone related behaviour is recorded by a skilled person and sperm count is determined for stallions. Important in the analysis of efficacy is the responder rate and duration of the obtained efficacy.

### Immunosterilization and Castration in Cattle

Entire male and female cattle are immunized with a formulation comprising Qβ-GnRH (comprising any peptide of SEQ ID NOs: 2-9 and 29-33) with or without adjuvant. Cattle were 9-12 months of age at the time of initial immunization. Each dose contains between 50 and 1000 μg Qβ-GnRH and given either subcutaneously, or intramuscular or through other routes of immunization. A boost immunization is given at 28 days after primary immunization. Blood samples are taken at monthly intervals after the boost immunization, and antibody titers are measured by ELISA. Alternatively one immunization only is given at 8 months. Testes size is monitored and anti-GnRH IgG titers are measured from serum.

The Qb-GnRH vaccine is given to bulls used for meat production as an alternative for surgical castration.

Female cattle (heifers) behaviour is monitored by daily inspection by trained farm staff and veterinarians. 8 weeks after boost immunization, behaviour is also monitored by fixing of Heat Mount Detector pads (Kamar Marketing Group Inc, Steamboat Springs, Colo., USA, dye releasing pads) to the rump of heifers. Mounting or riding behaviour (also called bulling) by cycling heifers will crush capsules of dye in the pads, which can be visualised from a distance. This usually only occurs when the standing heifer is receptive, i.e. in oestrus, and when the mounting heifer is also in oestrus. Thus the pads provide a useful continual monitor of oestrus in immunized heifers run with control unimmunized heifers.

### Example 13

### Cloning, Expression and Purification of the Modified VLP of AP205 Displaying GnRH Cloning of GnRH at the C-Terminus of AP205 Coat Protein

The DNA fragment coding for the GnRH peptide (EHWSYGLRPG, SEQ ID NO:1) was created by annealing two oligonucleotides—oligo 4.56 (gttccggaga acactggtcc tatggactca ggcctggtta atgcattg, SEQ ID NO:44) and oligo 4.57 (caatgcatta accaggcctg agtccatagg accagtgttc tccggaac, SEQ ID NO:45). The obtained fragment was digested with Kpn2I and Mph1103I and cloned in the same restriction sites into the vector pAP405-61 comprising a DNA encoding the AP205 core protein and a spacer sequence (GTAGGGSG) under the control of E. coli tryptophan operon promoter. The resulting construct AP205-489 has the following structure: AP205 coat protein—GTAGGGSG spacer—EHWSYGLRPG. The DNA sequence of the entire plasmid is depicted in SEQ ID NO:46. The AP205-GnRH fusion construct is represented by bp 131-580 of SEQ ID NO:46.

### Purification

Cells were lysed Cells were lysed by three freeze thaw cycles in a Tris-buffered lysis buffer containing 1 mg/ml lysozyme and 0.1% Tween 20 followed by ultrasonication. The lysate was clarified by centrifugation. The pellet was extracted with four portions of a buffer containing 7 M urea and 0.05 M Tris. The pooled supernatants were loaded on a Sepharose CL-2B column equilibrated in NET buffer (20 mM Tris-HCl, pH 7.8 with 5 mM EDTA and 150 mM NaCl), and rechromatographed on a sepharose 6B column. Capsid assembly was confirmed by EM analysis.

### Example 14

### Neutralising Antibody Response of Mice Immunized with AP205-GnRH VLPs

Immunization of Mice with AP205-GnRH VLPs for Suppression of Testicular Function

Recombinantly produced AP205-GnRH fusion VLPs were used for immunization. Eight week old mMale C57B16 mice (8 weeks old, five mice per group) were immunized with 50 μg of AP205-GnRH on day 0 and day 21. Control mice of the same age did not receive any vaccine. Anti-GnRH antibody titers and testosterone levels were measured in these mice. On day 70, mice were killed and testes weight determined.

### Anti-GnRH Antibody Titers in Mice

Serum was collected from immunized mice and control mice at various time points during the experiment. Anti-GnRH IgG antibody titer was determined by ELISA as follows. ELISA plates (Nunc Maxisorp) were coated with 10 μg/ml of CGG-GnRH (SEQ ID NO:2) coupled to RNase. Plates were blocked with 2% BSA and incubated with serial dilutions of mice sera. As a control, pre-immune sera was also tested. As a secondary antibody, 1:1000 dilution of goat anti-mouse IgG (H+L)−HRPO (Jackson ImmunoResearch Cat no 115-035-146) was used. After substrate addition and stopping the color reaction, optical density (OD) at 450 nm was determined on an ELISA reader (BioRad Benchmark). Using these data the serum dilution resulting in half the maximum OD450 was calculated.

Table A shows that in male mice immunized with AP205-GnRH, an average titer of 10876 was reached on day 21 and that a second immunization resulted in an average titer of 44047 on day 28. These results This clearly shows that AP205-GnRH is able to induce a high antibody titer against GnRH.

TABLE A
Anti-GnRH IgG antibody titers of male mice immunized with 50 μg
AP205-GnRH on day 0 and day 21. Titers are expressed as the
dilution resulting in 50% maximum binding. Averages of 5 mice
and standard deviations (sd) are indicated.

| Time-point of | 50% ODmax titer |
|---|---|
| bleeding | (±sd) |
| day 21 | 10876 ± 4446 |
| day 28 | 44047 ± 14831 |
| day 45 | 33306 ± 14722 |
| day 70 | 25958 ± 12196 |

### Testosterone Levels in Mice Serum

Serum was collected from immunized mice and control mice at various time points during the above described experiment. Using a Testosterone-ELISA (IBL, Hamburg, Germany) the testosterone levels in individual mice sera were determined.

Table B shows that in mice immunized with AP205-GnRH, the average testosterone level is greatly suppressed from day 28 to day 70 as compared to control mice. Control mice showed strong natural variation in testosterone levels with the average levels being approximately 5- to 50-fold higher than the levels in AP205-GnRH immunized mice. This clearly demonstrates neutralising activity of the induced antibody response.

TABLE B
Testosterone levels (ng/ml) in male mice immunized with 50 μg
AP205-GnRH on day 0 and day 21. Averages of 5 mice and
standard deviations (±sd) are indicated.

| Time-point of | AP205-GnRH | untreated |
|---|---|---|
| bleeding | testosterone ng/ml | testosterone ng/ml |
| day 0 | 2.05 ± 2.47 | 6.11 ± 6.35 |
| day 21 | 2.23 ± 3.75 | 2.41 ± 2.99 |
| day 28 | 0.86 ± 0.76 | 3.34 ± 5.9 |
| day 45 | 0.06 ± 0.09 | 3.63 ± 2.17 |
| day 70 | 0.82 ± 0.68 | 4.15 ± 3.95 |

### Testes Weight

Mice were sacrificed on day 70 and testes were removed and weighed, before fixing in 4% formaldehyde.

Table C shows the strongly reduced testes weight of AP205-GnRH immunized mice on day 70. On average a greater than 37% reduction in testes weight was obtained in immunized mice as compared to controls demonstrating neutralising activity of the induced anti-GnRH antibody response.

TABLE C
Testes weight of male mice immunized with AP205-GnRH and
sacrificed on day 70. Averages of 5 mice and standard
deviations (±sd) are indicated.

| Group | testes weight (gram) on day 70 |
|---|---|
| AP205-GnRH | 0.133 ± 0.034 |
| controls | 0.213 ± 0.026 |

### Example 15

### Immunization of Male Pigs with Qβ-CGG-GnRH and AP205-GnRH for the Prevention of Boar Taint in Meat

The Qβ-CGG-GnRH conjugate and the recombinantly produced AP205-GnRH fusion vaccines were evaluated for immunogenicity in male piglets which were 9 weeks of age at the first immunization and 13 weeks of age at the time-point of second immunization. Pigs received 400 μg of either Qβ control (n=2), or Qβ-CGG-GnRH conjugate (n=4), or AP205-GnRH fusion (n=2) with the adjuvant DEAE Dextran (DD), subcutaneously in the neck. A further two pigs were immunized subcutaneously with a high dose of Qβ-GnRH (1.2 mg) in the presence of DEAE Dextran at week 13. Anti-GnRH IgG antibody titres were determined by ELISA throughout the course of the experiment. Animals were sacrificed at week 26.

Table D shows that the Qβ-CGG-GnRH and AP205-GnRH vaccines both result in appreciable anti-GnRH IgG antibody titers in week 13 after only priming and that upon second immunization, average anti-GnRH titers are boosted to over 1000 in week 16 and are still detectable at week 26. Immunization with a high dose of Qβ-CGG-GnRH at week 13 also results in high anti-GnRH antibody titers at week 16 and shows an appreciable anti-GnRH antibody titer at week 26.

TABLE D
Anti-GnRH IgG antibody titers of male pigs immunized with either
Qβ-CGG-GnRH or AP205-GnRH. Titers are expressed as the dilution resulting in
50% maximum binding. Group averages, standard deviations (sd) and numbers of
animals (n) are indicated.

| vaccine | Week 9 | Week 13 | Week 16 | Week 18 | Week 23 | Week 26 |
|---|---|---|---|---|---|---|
| Qβ + DD (n = 2) | 20 ± 0.1 | 31 ± 3.5 | 25 ± 6.7 | 20 ± 0 | 20 ± 0 | 23 ± 0.1 |
| Qβ-CGG-GnRH + DD (n = 4) | 24 ± 4 | 351 ± 90 | 1119 ± 259 | 773 ± 282 | 264 ± 134 | 218 ± 126 |
| AP205-GnRH + DD (n = 2) | 20 ± 0 | 450 ± 415 | 1105 ± 701 | 726 ± 375 | 204 ± 75 | 139 ± 36 |
| Qβ-CGG-GnRH 1.2 mg + DD | 20 ± 0 | 21 ± 1.0 | 1168 ± 824 | 987 ± 254 | 287 ± 118 | 183 ± 63 |
| (n = 2) | | | | | | |

### Testes Weight

Pigs were sacrificed at 26 weeks of age and testes plus epididymis were removed and weighed.

Table E shows the strongly reduced testes weight in some of the pigs that received a subcutaneous prime and boost immunization with either Qb-CGG-GnRH or AP205-GnRH as compared to the testes weight of control animals. This clearly shows neutralising activity of the induced antibody response.

TABLE E
Testes weights (including epididymis) of pigs at 26 weeks of age
after immunization with either Qb-CGG-GnRH or AP205-GnRH.
Testes weights are indicated for each individual animal.

| | Testes weight |
|---|---|
| Group | (incl. epididymis) in grams |
| Qb + DD (n = 2) | 632, 633 |
| Qb-CGG-GnRH + DD (n = 4) | 111, 475, 485, 871 |
| AP205-GnRH + DD (n = 2) | 189, 705 |

### Example 16

### Immunization of Male Pigs with Qβ-CGG-GnRH Causes a Decrease of Serum Testosterone Levels

In a separate experiment two pigs were immunized subcutaneously with 400 μg Qβ-CGG-GnRH in the presence of DEAE-Dextran at 23 weeks of age after having received an initial priming immunization. Two other pigs were immunized with control VLP. Blood was taken from animals at 23 and 26 weeks of age.

Table F shows that immunization with Qβ-CGG-GnRH in the presence of DEAE-Dextran at week 23 results in a marked decrease of testosterone production in week 26 as compared to the animals receiving control VLP. This demonstrates neutralising activity of the anti-GnRH antibodies raised after Qβ-CGG-GnRH immunization.

TABLE F
Serum testosterone levels after immunization of pigs with
Qβ-CGG-GnRH. Testosterone levels are indicated for
individual animals.

| Testosterone levels | | |
|---|---|---|
| (ng/ml) | week 23 | week 26 |
| Qβ-CGG-GnRH | 0.87 | 0.29 |
| Qβ-CGG-GnRH | 2.19 | 0.28 |
| Control VLP | 1.22 | 1.72 |
| Control VLP | 3.15 | 2.32 |

What is claimed is:

1. A composition comprising:
(a) a virus like particle (VLP), and
(b) at least one GnRH peptide;
wherein a) and b) are linked with one another.

2. The composition of claim 1, wherein said at least one GnRH peptide is selected from the group consisting of
(a) GnRH1-10 (SEQ ID NO: 1),
(b) GnRH 2-10 (SEQ ID NO: 6),
(c) GnRH 3-10 (SEQ ID NO: 7),
(d) GnRH 4-10 (SEQ ID NO: 8),
(e) GnRH 5-10 (SEQ ID NO: 9),
(f) GnRH 6-0 (SEQ ID NO: 43),
(g) GnRH 1-9 (SEQ ID NO: 29),
(h) GnRH 1-8 (SEQ ID NO: 30),
(i) GnRH 1-7 (SEQ ID NO: 31),
(j) GnRH 1-6 (SEQ ID NO: 32),
(k) GnRH 1-5 (SEQ ID NO: 33),
(l) SEQ ID NO: 28,
(m) SEQ ID NO:34,
(n) SEQ ID NO:35,
(o) SEQ ID NO: 36, and
(p) fragments of any of the sequences of (a) to (o).

3. The composition of claim 1, wherein said at least one GnRH peptide is GnRH 1-10 (SEQ ID NO: 1).

4. The composition of any one of claims 1 to 3, wherein the composition forms an ordered and repetitive antigen array.

5. The composition of any one of claims 1 to 4, wherein the VLP (a) and the at least one GnRH-peptide (b) are covalently linked.

6. The composition of claim 5, wherein the VLP (a) is linked with the at least one GnRH-peptide (b) through at least one non-peptide bond, and wherein preferably said at least one GnRH-peptide is linked to said VLP via its N-terminus.

7. The composition of any one of claims 1 to 5, wherein said GnRH-peptide is fused to said VLP, and wherein preferably said GnRH-peptide is fused via its N-terminus to the VLP.

8. The composition of any one of the preceding claims, wherein said VLP comprises at least one first attachment site, wherein said at least one GnRH peptide comprises at least one second attachment site; and wherein said VLP and said at least one GnRH-peptide are linked through said at least one first and said at least one second attachment site.

9. The composition of claim 8, wherein the first attachment site comprises, or preferably is, an amino group, preferably an amino group of a lysine.

10. The composition of claim 8 or 9, wherein said second attachment site comprises, or preferably is, a sulfhydryl group, preferably a sulfhydryl group of a cysteine.

11. The composition of any one of the preceding claims further comprising a linker (c) between said VLP and said at least one GnRH-peptide.

12. The composition of claim 11, wherein said linker (c) comprises or consists of said second attachment site.

13. The composition of claim 11 or 12, wherein said linker (c) does not essentially affect the immune response against GnRH.

14. The composition of any one of claims 11 to 13, wherein said linker comprises, consists essentially of, or consists of less than 5, preferably less than 4, more preferably less than 3, even more preferably less than 2 amino acids.

15. The composition of any one of claims 11 to 14, wherein said linker is attached at the N-terminus of said at least one GnRH peptide.

16. The composition of any one of claims 11 to 15, wherein said linker is selected from the group consisting of;
(a) C;
(b) CG;
(c) CGG;
(d) GC; and
(e) GGC.

17. The composition of any one of claims 11 to 16, wherein said linker is C.

18. The composition of any one of claims 8 to 17, wherein said GnRH peptide with said second attachment site has an amino acid sequence selected from the group consisting of:

| | |
|---|---|
| (a) CGGEHWSYGLRPG; | (SEQ ID NO: 2) |
| (b) EHWSYGLRPGGGC; | (SEQ ID NO: 3) |
| (c) CEHWSYGLRPG; | (SEQ ID NO: 4) |
| and | |
| (d) EHWSYGLRPGC. | (SEQ ID NO: 5) |

19. The composition of claim 18, wherein said GnRH peptide with said second attachment site has the amino acid sequence of SEQ ID NO: 4.

20. The composition of any one of the preceding claims, wherein said VLP is a recombinant VLP.

21. The composition of any one of the preceding claims, wherein said VLP comprises recombinant proteins, or fragments thereof, selected from the group consisting of:
(a) recombinant proteins of RNA-phages;
(b) recombinant proteins of bacteriophages;
(c) recombinant proteins of Hepatitis B virus;
(d) recombinant proteins of measles virus;
(e) recombinant proteins of Sindbis virus;
(f) recombinant proteins of Rotavirus;
(g) recombinant proteins of Foot-and-Mouth-Disease virus;
(h) recombinant proteins of Retrovirus;
(i) recombinant proteins of Norwalk virus;
(j) recombinant proteins of Alphavirus;
(k) recombinant proteins of human Papilloma virus;
(l) recombinant proteins of Polyoma virus;
(m) recombinant proteins of Ty; and
(n) fragments of any of the recombinant proteins from (a) to (n).

22. The composition of any one of the preceding claims, wherein said VLP comprises, or alternatively consists of, recombinant proteins, or fragments thereof, of a RNA-phage.

23. The composition of claim 20, wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(j) bacteriophage f2;
(k) bacteriophage PP7; and
(l) bacteriophage AP205.

24. The composition of any one of the preceding claims, wherein said VLP comprises, or alternatively consists of, recombinant coat proteins, or fragments thereof, of RNA-phage Qβ.

25. The composition of any one of the preceding claims, wherein said VLP comprises, or alternatively consists of, recombinant coat proteins, or fragments thereof, of RNA-phage fr.

26. The composition of any one of the preceding claims, wherein said VLP comprises, or alternatively consists of, recombinant coat proteins, or fragments thereof, of RNA-phage AP205.

27. The composition of any of the preceding claims, wherein the recombinant proteins comprise, or alternatively consist essentially of, or alternatively consist of coat proteins of RNA phages.

28. The composition of claim 27, wherein said coat protein of RNA phages having an amino acid are selected from the group comprising or, alternatively consisting of:
(a) SEQ ID NO: 10;
(b) a mixture of SEQ ID NO: 10 and SEQ ID NO: 11;
(c) SEQ ID NO: 12;
(d) SEQ ID NO: 13;
(e) SEQ ID NO: 14;
(f) SEQ ID NO: 15;
(g) a mixture of SEQ ID NO: 15 and SEQ ID NO: 16;
(h) SEQ ID NO: 17;
(i) SEQ ID NO: 18;
(j) SEQ ID NO: 19;
(k) SEQ ID NO: 20;
(l) SEQ ID NO: 21;
(m) SEQ ID NO:22; and
(n) SEQ ID NO: 39.

29. The composition of any one of the preceding claims, wherein the recombinant proteins comprise, or alternatively consist essentially of, or alternatively consist of mutant coat proteins of RNA phages.

30. The composition of claim 29, wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(j) bacteriophage f2;
(k) bacteriophage PP7; and
(l) bacteriophage AP205.

31. The composition of any one of claim 29 or 30, wherein said mutant coat proteins of said RNA phage have been modified by removal of at least one lysine residue by way of substitution.

32. The composition of any one of claim 29 or 30, wherein said mutant coat proteins of said RNA phage have been modified by addition of at least one lysine residue by way of substitution.

33. The composition of any one of claim 29 or 30, wherein said mutant coat proteins of said RNA phage have been modified by deletion of at least one lysine residue.

34. The composition of any one of claims 29 to 30, wherein said mutant coat proteins of said RNA phage have been modified by addition of at least one lysine residue by way of insertion.

35. A vaccine composition comprising a composition of any one of claims 1 to 34.

36. The vaccine composition of claim 35, wherein said vaccine composition is devoid of an adjuvant.

37. The vaccine composition of claim 35, further comprising an adjuvant.

38. The vaccine composition of any one of claims 35-37, wherein said composition comprises recombinant proteins or fragments thereof, of RNA-phage Qβ.

39. A method of immunization comprising administering a composition of any of the claims 1-32 or a vaccine composition of any of the claim 35-37 to an animal or human.

40. A pharmaceutical composition comprising:
(a) the composition of any of claims 1 to 34 or the vaccine composition of any of the claim 35-38; and
(b) an acceptable pharmaceutical carrier.

41. The pharmaceutical composition of claim 40 further comprising an adjuvant

42. The pharmaceutical composition of claim 40, wherein said pharmaceutical composition is devoid of an adjuvant.

43. A method of producing the composition of any one of claims 1 to 34 comprising:
(a) providing a VLP with at least one first attachment site;
(b) providing a GnRH peptide with at least one second attachment site, wherein said second attachment site is capable of association to said first attachment site; and
(c) combining said VLP and said GnRH peptide to produce a composition, wherein said GnRH peptide and said VLP interact through said association.

44. The method of claim 43, wherein the composition forms an ordered and repetitive antigen array.

45. Composition of any one of claims 1 to 34 for use as a medicament.

46. Use of the composition of any one of claims 1 to 34 for the manufacture of a medicament or vaccine for treating or modulating a disease or condition in an animal associated with GnRH, preferably wherein said disease or condition is selected from the group consisting of fertility, gonadal steroid hormone dependent cancer, prostate cancer, boar taint in pork, meat quality of male animals kept for meat production, gonadal steroid hormone related behaviour in animals, and reproduction in wild life animals, wherein preferably said animals kept for meat production are rams, boars, or bulls.

47. A method of treating a GnRH associated disease or condition comprising administering the composition of any of the claim 1-34, the vaccine composition of any of the claim 35-38 or the pharmaceutical composition of any of the claim 40-42 to an animal or human.

48. The method of claim 47, wherein said disease or condition is selected from the group consisting of fertility, gonadal steroid hormone dependent cancer, prostate cancer, boar taint in pork, beef or sheep, meat quality of male animals kept for meat production, gonadal steroid hormone related behaviour in animals, and reproduction in wild life animals, wherein preferably said animals kept for meat production are rams, boars, or bulls.

49. The method of claim 47, wherein said disease or condition is fertility.

50. The method of any one of claims 47 to 50, wherein said administering is effected in an animal.

51. The method of any one of claims 47 to 50, wherein said animal is a mammal, preferably a pet or a horse.

52. The method of any one of claims 47 to 51, wherein said animal is a female.

53. The method of any one of the claims 47 to 52, wherein said administering is effected by at most a first administration, a second and a third administration of said composition, said vaccine composition, or said pharmaceutical composition.

54. The method of any one of the claims 47 to 52, wherein said administering is effected by at most a first administration and a second administration of said composition, said vaccine composition, or said pharmaceutical composition.

55. The method of any one of the claims 47 to 52, wherein said administering is effected by only a first single administration of said composition, said vaccine composition, or said pharmaceutical composition.

56. The method of claim 47, wherein said administering is effected in a pig, cattle or sheep, and wherein said condition is boar taint in pork, beef or sheep.

57. The method of claim 56, wherein said administering is effected by at most a first administration, a second and a third administration of said composition, said vaccine composition, or said pharmaceutical composition.

58. The method of claim 56 wherein said administering is effected by a at most first administration and a second administration of said composition, said vaccine composition, or said pharmaceutical composition.

59. The method of claim 56, wherein said administering is effected by only a first single administration of said composition, said vaccine composition, or said pharmaceutical composition.

60. The method of any one of the claims 57 to 59, wherein said first administration is effected 4 to 8 weeks prior to the slaughter of said pig, cattle or sheep.

61. A method of reducing boar taint in meat comprising administering the composition of any of the claim 1-34, the vaccine composition of any of the claim 35-38 or the pharmaceutical composition of any of the claim 40-42 to an animal

62. The method of claim 61 wherein the animal is a male.

63. The method of any one of claims 61 and 62 wherein the animal is a pig, cattle or sheep, preferably a pig.

64. The method of any one of claims 61 to 63 wherein said administering is effected by at most a first administration, a second and a third administration of said composition, said vaccine composition, or said pharmaceutical composition.

65. The method of any one of claims 61 to 63 wherein said administering is effected by at most a first administration and a second administration of said composition, said vaccine composition, or said pharmaceutical composition.

66. The method of any one of claims 61 to 63 wherein said administering is effected by only a first single administration of said composition, said vaccine composition, or said pharmaceutical composition.

67. The method of claim 66 wherein said first administration is effected 4 to 8 weeks before slaughter of said pig, cattle or sheep.

68. A method of preventing, reducing or eliminating the fertility of an animal comprising administering the composition of any of the claim 1-34, the vaccine composition of any of the claim 35-38 or the pharmaceutical composition of any of the claim 40-42 to said animal.

69. The method of claim 68 wherein said animal is a female.

70. The method of any one of claims 68 and 69 wherein said animal is a mammal, preferably a pet or a horse.

71. The method of any one of claims 68 to 70 wherein said animal is a dog, a cat or a rodent.

72. The method of any one of claims 68 to 71 wherein said administering is effected by at most a first administration, a second and a third administration of said composition, said vaccine composition, or said pharmaceutical composition.

73. The method of any one of claims 68 to 71 wherein said administering is effected by st most a first administration and a second administration of said composition, said vaccine composition, or said pharmaceutical composition.

74. The method of any one of claims 68 to 71 wherein said administering is effected by only a first single administration of said composition, said vaccine composition, or said pharmaceutical composition.


SEQUENCE LISTING (46 sequences)

SEQ ID NO: 1 - protein, 10 aa, mammalian
EHWSYGLRPG

SEQ ID NO: 2 - protein, 13 aa, Artificial Sequence
  GnRH analogue CGG-GnRH
CGGEHWSYGLRPG

SEQ ID NO: 3 - protein, 13 aa, Artificial Sequence
  GnRH analogue GnRH-GGC
EHWSYGLRPGGGC

SEQ ID NO: 4 - protein, 11 aa, Artificial Sequence
  GnRH analogue C-GnRH
CEHWSYGLRPG

SEQ ID NO: 5 - protein, 11 aa, Artificial Sequence
  GnRH analogue GnRH-C
EHWSYGLRPGC

SEQ ID NO: 6 - protein, 9 aa, Artificial Sequence
  GnRH analogue GnRH 2-10
HWSYGLRPG

SEQ ID NO: 7 - protein, 8 aa, Artificial Sequence
  GnRH analogues GnRH 3-10
WSYGLRPG

SEQ ID NO: 8 - protein, 7 aa, Artificial Sequence
  GnRH analogue GnRH 4-10
SYGLRPG

SEQ ID NO: 9 - protein, 6 aa, Artificial Sequence
  GnRH analogue GnRH 5-10
YGLRPG

SEQ ID NO: 10 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 11 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 12 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 13 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 14 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 15 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 16 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 17 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 18 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 19 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 20 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 21 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 22 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 23 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 24 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 25 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 26 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 27 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 28 - protein, 10 aa, Artificial Sequence
  GnRH analogue GnRH 1Q-10
QHWSYGLRPG

SEQ ID NO: 29 - protein, 9 aa, Artificial Sequence
  GnRH analogue GnRH 1-9
EHWSYGLRP

SEQ ID NO: 30 - protein, 8 aa, Artificial Sequence
  GnRH analogue GnRH 1-8
EHWSYGLR

SEQ ID NO: 31 - protein, 7 aa, Artificial Sequence
  GnRH analogue GnRH 1-7
EHWSYGL

SEQ ID NO: 32 - protein, 6 aa, Artificial Sequence
  GnRH analogue GnRH 1-6
EHWSYG

SEQ ID NO: 33 - protein, 5 aa, Artificial Sequence
  GnRH analogue GnRH 1-5
EHWSY

SEQ ID NO: 34 - protein, 20 aa, Artificial Sequence
  GnRH analogue tandem sequence
EHWSYGLRPGEHWSYGLRPG

SEQ ID NO: 35 - protein, 20 aa, Artificial Sequence
  GnRH analogue tandem sequence
EHWSYGLRPGQHWSYGLRPG

SEQ ID NO: 36 - protein, 20 aa, Artificial Sequence
  GnRH analogue tandem sequence
QHWSYGLRPGQHWSYGLRPG

SEQ ID NO: 37 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 38 - protein, 152 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVV

SEQ ID NO: 39 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 40 - protein, 5 aa, Artificial Sequence
  Linker
GGKGG

SEQ ID NO: 41 - protein, 4 aa, Artificial Sequence
  C.terminal linker
GGCG

SEQ ID NO: 42 - protein, 10 aa, Artificial Sequence
  GnRH analogue GnRH 1-10 lys
EHWSYKLRPG

SEQ ID NO: 43 - protein, 5 aa, Artificial Sequence
  GnRH analogue GnRH 6-10
GLRPG

SEQ ID NO: 44 - DNA, 48 bp, Artificial Sequence
  oligonucleotide
gttccggagaacactggtcctatggactcaggcctggttaatgcattg

SEQ ID NO: 45 - DNA, 48 bp, Artificial Sequence
  oligonucleotide
caatgcattaaccaggcctgagtccataggaccagtgttctccggaac

SEQ ID NO: 46 - DNA, 3700 bp, Artificial Sequence
  Vector Sequence
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggaaccgcaggaggcggttccggagaacactggtcctatggactcaggcctggttaatgcatgtctaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt